(19) United States
(12) Patent Application Publication
(10) Pub. No.: US 2005/0074754 A1
(43) Pub. Date: Apr. 7, 2005

(54) Novel protein and use thereof

(76) Inventors: Shoichi Okubo, Ushiku-shi (JP); Kuniko Kikuchi, Toride-shi (JP); Yasushi Shintani, Toyonaka-shi (JP)

Correspondence Address: TAKEDA PHARMACEUTICALS NORTH AMERICA, INC;INTELLECTUAL PROPERTY DEPARTMENT, 475 HALF DAY ROAD, SUITE 500, LINCOLNSHIRE, IL, 60069, US

(21) Appl. No.: 10/258,182

(22) PCT Filed: Apr. 27, 2001
(86) PCT No.: PCT/JP01/03672

(30) Foreign Application Priority Data
Apr. 27, 2000 (JP) 2000-127547
Mar. 8, 2001 (JP) 2001-64862

(51) Int. Cl. C12Q 1/68; C07H 21/04; C07K 14/705; C07K 16/28

(52) U.S. Cl. 435/6; 435/69.1; 435/320.1; 435/325; 530/350; 530/388.22; 536/23.5

(57) ABSTRACT

A novel gene likely inhibiting the onset and progress of cancer. A protein having an amino acid sequence which is the same or substantially the same as the amino acid sequence represented by SEQ ID NO:4 or its salt; a polynucleotide encoding the same; and medicinal use, etc. thereof are provided.

### TECHNICAL FIELD

This invention relates to a novel protein and use thereof

### BACKGROUND ART

For the functional actions of cells, it is essential that individual cells constituting many living organisms including human beings recognize intracellular environments accurately to cope rapidly therewith. For this purpose, various molecules present on the surface of cells have important functions, and in particular many protein molecules including receptors play the central role. For example, many Tyr kinase-type receptors corresponding to extracellular ligands function by phosphorylating specific intracellular proteins. Further, some surface proteins expressed in a certain kind of leukocytes participate in immunoreaction in the body by acting on other cells through intercellular interaction. Many of these protein molecules are present on and fixed in some forms to the cell membrane. However, some are released extracellularly by an enzyme or via a certain other mechanism, to function as secretory proteins. There are diverse secretory proteins including those enhancing or antagonizing original functions or those having completely different functions. For example, a large number of secretory receptors compete for ligands with receptors on the membrane. Further, molecules such as CD55 and CD59, whether secretory type or membrane-bound type, work for repression of the complement system (Immunology Today 20, 576-582 (1999)).

On one hand, a large number of genes for proteins such as cell growth factors or their receptors, transcription factors, and proteins involved in signal transduction are found as cancer-related genes. For example, there are PDGF relating to brain tumor (Nature 362, 801 (1997)), c-myc and N-myc relating to breast cancer, stomach cancer and neuroblastoma, and Ki-ras and N-ras relating to colon cancer and leukemia. These genes were identified from information on the sequences of specific proteins and genes found upon transformation of specific cells and tissues, and are not commonly expressed on all or many cancer cells or tissues. Further, the immune systems for mammals including humans are very complicated, and the elucidation of the mechanism of development and progress of all cancers by a single gene product is expected to be difficult.

Accordingly, cancer-related genes involved in cancer development and progress or genes coding for a group of proteins called cancer antigen molecules whose expression is enhanced upon transformation attract attention in recent years, and there is need for discovery of a new gene which may directly or indirectly promote cancer development and progress by particularly participating in some way in the immune system.

### SUMMARY OF INVENTION

The present inventors made extensive study to solve the problem described above, and as a result, they found a novel gene coding for a membrane-bound and/or secretory protein having a hydrophobic amino acid cluster in both N- and C-terminals on a predicted amino acid sequence.

On the basis of these findings, the present inventors made further study, to complete the present invention.

That is, the present invention provides:

1. A protein comprising an amino acid sequence identical or substantially identical with the amino acid sequence set forth in SEQ ID NO:4 or a salt thereof,

2. A partial peptide of the protein described in item 1, or an amide, an ester or a salt thereof,

3. A DNA comprising a DNA coding for the protein described in item 1;

4. The DNA according to item 3, which comprises the nucleotide sequence set forth in SEQ ID NO:3,

5. A DNA comprising a DNA coding for the partial peptide described in item 2,

6. A recombinant vector comprising the DNA described in item 3 or 5;

7. A transformant transformed with the recombinant vector described in item 6,

8. A process for producing the protein described in item 1 or a salt thereof, or the partial peptide described in item 2 or an amide, an ester or a salt thereof, which comprises culturing the transformant described in item 7, and obtaining the protein of item 1, or the partial peptide of item 2,

9. An antibody against the protein described in item 1 or a salt thereof, or the partial peptide described in item 2 or an amide, an ester or a salt thereof,

10. A diagnostic composition comprising the DNA described in item 3 or 5, or the antibody described in item 9,

11. A pharmaceutical composition comprising the protein described in item 1 or a salt thereof, or the partial peptide described in item 2 or an amide, an ester or a salt thereof, or the antibody described in claim 9,

12. A pharmaceutical composition according to item 11, for preventing and treating cancers,

13. A method of screening a compound or a salt thereof promoting or inhibiting an activity of the protein described in item 1 or a salt thereof, or the partial peptide described in item 2 or an amide, an ester or a salt thereof, which comprises using the protein described in item 1 or a salt thereof, or the partial peptide described in item 2 or an amide thereof, an ester thereof or a salt thereof,

14. A kit for screening a compound or a salt thereof promoting or inhibiting an activity of the protein described in item 1 or a salt thereof, or the partial peptide described in item 2 or an amide, an ester or a salt thereof, which comprises the protein described in item 1 or a salt thereof, or the partial peptide described in item 2 or an amide thereof, an ester thereof or a salt thereof,

15. A compound or a salt thereof promoting an activity of the protein described in item 1 or a salt thereof, or the partial peptide described in item 2 or an amide, an ester or a salt thereof, which is obtainable by the screening method described in item 13 or the screening kit described in item 14,

16. A pharmaceutical composition comprising the compound described in item 15 or a salt thereof,

17. The pharmaceutical composition according to item 16, for preventing and treating cancer,

18. Use of (i) the protein described in item 1 or a salt thereof, or the partial peptide described in item 2 or an amide, an ester or a salt thereof, or (ii) the compound or a salt thereof promoting an activity of the protein described in item 1 or a salt thereof, or the partial peptide described in item 2 or an amide, an ester or a salt thereof, which is obtained by the screening method described in item 13 or the screening kit described in item 14, for producing a pharmaceutical composition having an anticancer action,

19. A method of preventing and treating cancer, which comprises administering to mammals (i) the protein described in item 1 or a salt thereof, or the partial peptide described in item 2 or an amide, an ester or a salt thereof, or (ii) the compound or a salt thereof promoting an activity of the protein described in item 1 or a salt thereof, or the partial peptide described in item 2 or an amide, an ester or a salt thereof, which is obtained by the screening method described in item 13 or the screening kit described in item 14.

20. A compound or a salt thereof inhibiting an activity of the protein described in item 1 or a salt thereof, or the partial peptide described in item 2 or an amide, an ester or a salt thereof, which is obtained by the screening method described in item 13 or the screening kit described in item 14,

21. A pharmaceutical composition comprising the compound described in item 20 or a salt thereof,

22. The pharmaceutical composition according to item 21, which is an immunosupressant or an antiinflammatory composition,

23. Use of (i) the compound or a salt thereof inhibiting the activity of the protein described in item 1 or a salt thereof, or the partial peptide described in item 2 or an amide, an ester or a salt thereof, which is obtained by the screening method described in item 13 or the screening kit described in item 14, or (ii) the antibody described in item 9, for producing a pharmaceutical composition having an immunosuppressive or antiinflammatory action, and

24. A method of immunorepression or a method of treating inflammations, which comprises administering to mammals the compound or a salt thereof inhibiting the activity of the protein described in item 1 or a salt thereof, or the partial peptide described in item 2 or an amide thereof, an ester thereof or a salt thereof, which is obtained by the screening method described in item 13 or the screening kit described in item 14, or the antibody described in item 9.

Further, this invention provides:

25. The protein according to item 1 or a salt thereof, wherein the amino acid sequence substantially identical with the amino acid sequence set forth in SEQ ID NO:4 is an amino acid sequence having about 70% or more, preferably about 80% or more, more preferably about 90% or more, still more preferably about 95% or more homology with the amino acid sequence set forth in SEQ ID NO:4,

26. The protein according to item 1 or a salt thereof, wherein the amino acid sequence substantially identical with the amino acid sequence set forth in SEQ ID NO:4 is (i) an amino acid sequence set forth in SEQ ID NO:4 wherein 1 to 5 (preferably 1 to 3) amino acids are deleted, (ii) an amino acid sequence set forth in SEQ ID NO:4 wherein 1 to 10 (preferably 1 to 5 (more preferably 1 to 3)) amino acids are added, (iii) an amino acid sequence set forth in SEQ ID NO:4 wherein 1 to 5 (preferably 1 to 3) amino acids are replaced by other amino acids, or (iv) a combination of the above amino acid sequences,

27. The screening method described in item 13, which comprises measuring an activity of the protein described in item 1 or a salt thereof, or the partial peptide described in item 2 or an amide, an ester or a salt thereof, and comparing the activity between in the case (i) where a substrate is contacted with the protein described in item 1 or a salt thereof, or the partial peptide described in item 2 or an amide, an ester or a salt thereof and in the case (ii) where the substrate and a test compound are contacted with the protein described in item 1 or a salt thereof, or the partial peptide described in item 2 or an amide, an ester or a salt thereof,

28. A method of quantifying the protein described in item 1, or the partial peptide described in item 2 or a salt thereof in a test solution, which comprises competitively reacting a test solution and the labeled protein described in item 1 or a salt thereof, or the labeled partial peptide described in item 2 or an amide, an ester or a salt thereof with the antibody described in item 9, and then measuring a proportion of the labeled protein described in item 1 or a salt thereof, or the labeled partial peptide described in item 2 or an amide, an ester or a salt thereof which is bound to the antibody, and

29. A method of quantifying the protein described in item 1, or the partial peptide described in item 2 or a salt thereof in a test solution, which comprises reacting a test solution simultaneously or successively with the antibody of item 9 insolubilized on a carrier and the labeled antibody of item 9, and then measuring an activity of the label on the insolubilizing carrier.

### BRIEF DESCRIPTION OF DRAWINGS

FIG. 1 is a Western blotting showing expression of CSP-FLAG in COS-7 cells.

FIG. 2A is a histogram of FITC when a concentrate of a supernatant culture of CSP2-Fc-expressing COS7 cells was added to CHO-K1/hNKG2D-11 cells; B is a histogram of FITC when a concentrate of a supernatant culture of CSP2-Fc-expressing COS7 cells obtained in Example 6 was added to CHO-K1 cells (control); C is a histogram of FITC when a concentrate of a supernatant culture of a Mock plasmid-containing COS7 cells was added to CHO-K1/hNKG2D-11 cells; and D is a combination of histograms A to C.

### BEST MODE OF THE INVENTION

The protein having an amino acid sequence identical or substantially identical with the amino acid sequence set forth in SEQ ID NO:4 in the present invention (referred to hereinafter as the protein of the invention) may be a protein derived from cells (e.g., hepatic cells, spleen cells, nerve cells, glia cells, pancreatic β-cells, bone marrow cells, mesangial cells, Langerhans cells, epidermal cells, epithelial cells, endothelial cells, fibroblasts, fibrous cells, muscular cells, adipocytes, immunocytes (e.g., macrophage, T cells, B cells, natural killer cells, mast cells, neutrophils, basophils, acidophils, monocytes), megakaryocytes, synovial membrane cells, cartilage cells, bone cells, osteoblasts, osteoclasts, mammary gland cells, hepatic cells or interstitial cells, or their precursor cells, stem cells or cancer cells) or any tissues having such cells, for example, the brain and each site of the brain (e.g., olfactory bulb, tonsil nuclei, cerebrum basal ganglion, hippocampus, thalamus, hypothalamus, cerebral cortex, medulla bulb, cerebellum), spinal cord, pituitary, stomach, pancreas, kidney, liver, gonads, thyroid glands, galls, bone marrow, adrenals, skin, muscles, lungs, digestive tracts (e.g., large and small intestines), blood vessels, heart, thymus, spleen, salivary glands, peripheral blood, prostate, testicles, ovary, placenta, uterus, bone, joints, skeletal muscles, or corpuscle cells or cultured cells thereof (for example, MEL, M1, CTLL-2, HT-2, WEHI-3, HL-60, JOSK-1, K562, ML-1, MOLT-3, MOLT-4, MOLT-10, CCRF-CEM, TALL-1, Jurkat, CCRT-HSB-2, KE-37, SKW-3, HUT-78, HUT-102, H9, U937, THP-1, HEL, JK-1, CMK, KO-812, MEG-01 etc.) from humans or warm-blooded animals (e.g., guinea pig, rat, mouse, chicken, rabbit, pig, sheep, cow, monkey etc.), as well as a synthetic protein.

The amino acid sequence substantially identical with the amino acid sequence set forth in SEQ ID NO:4 includes those amino acid sequences having about 70% or more, preferably about 80% or more, more preferably about 90% or more, still more preferably about 95% or more homology with the amino acid sequence set forth in SEQ ID NO:4.

The protein of the invention having an amino acid sequence substantially identical with the amino acid sequence set forth in SEQ ID NO:4 is preferably e.g. a protein having an amino acid sequence substantially identical with the amino acid sequence set forth in SEQ ID NO:4 and having substantially identical properties with those of a protein having the amino acid sequence set forth in SEQ ID NO:4.

“The substantially identical properties” means, for example, the activity of binding to NKG2D and the activation of immunocytes.

The term “substantially identical” means that between those proteins in question, their properties are identical qualitatively (e.g. physiochemically or pharmacologically). It follows that between those proteins in question, their qualitative properties such as activity of binding to NKG2D and activation of immunocytes are preferably identical (e.g., about 0.1- to 100-fold, preferably about 0.5- to 10-fold, more preferably about 0.5- to 2-fold), while their quantitative properties such as degree of these activities and molecular weight may be different.

The activity of binding to NKG2D may be measured by a method known per se, for example ELISA etc. Alternatively, the activity may also be measured according to a screening method described below.

Measurement of the activation of immunocytes can be carried out according to a method known per se, and for example, there is a method wherein growth of immunocytes is examined as an indicator of the activation of immunocytes. Specifically, this method comprises measuring intracellular DNA synthesis, where DNA synthesis can usually be quantified in terms of incorporation of thymidine or its derivative. That is, there is a method wherein incorporation of [³H]thymidine is quantified in terms of its radioactivity as an indicator or a method wherein incorporation of bromodeoxiuridine (BrdU) i.e. a derivative of thymidine is quantified by using an antibody specific to BrdU. In an alternative method, production of various cytokines accompanying the activation of immunocytes may be examined. Specifically, interleukins (IL-1, IL-2, IL-3I, L-4 etc.) and interferons (alpha, beta, gamma) TNF, GM-CSF, and various chemokines to be secreted into a medium or serum upon activation of immunocytes are measured by using antibodies specific thereto.

The protein of the invention includes e.g. proteins called mutein containing (1) an amino acid sequence set forth in SEQ ID NO:4 wherein 1 to 5 (preferably 1 to 3) amino acids are deleted, (2) an amino acid sequence set forth in SEQ ID NO:4 wherein 1 to 10 (preferably 1 to 5 (more preferably 1 to 3)) amino acids are added, (3) an amino acid sequence set forth in SEQ ID NO:4 wherein 1 to 5 (preferably 1 to 3) amino acids are inserted, (4) an amino acid sequence set forth in SEQ ID NO:4 wherein 1 to 5 (preferably 1 to 3) amino acids are replaced by other amino acids, or (5) a combination of these amino acid sequences.

If the amino acid sequence has an insertion, deletion or substitution as described above, the position of the insertion, deletion or substitution is not particularly limited.

In this specification, the N-terminal (amino terminal) of the protein is placed in the left and the C-terminal (carboxyl terminal) in the right in accordance with a peptide notation system. The protein of the invention, including the protein having the amino acid sequence set forth in SEQ ID NO:4, usually has a carboxyl group (—COOH) or carboxylate (—COO—) at the C-terminal, but may have an amide (—CONH) or ester (—COOR) at the C-terminal.

The group R in the ester includes e.g. C₁₋₆ alkyl groups such as methyl, ethyl, n-propyl, isopropyl and n-butyl, C₃₋₈ cycloalkyl groups such as cyclopentyl and cyclohexyl, C₆₋₁₂ aryl groups such as phenyl and α-naphthyl, and C₇₋₁₄ aralkyl groups, for example, phenyl-C₁₋₂ alkyl groups such as benzyl and phenetyl and α-naphthyl-C₁₋₂ alkyl groups such as α-naphthylmethyl, as well as a pivaloyloxymethyl group used generally for oral administration.

When the protein of the invention has a carboxyl group (or carboxylate) at other position than the C-terminal, the protein of the invention includes those proteins wherein said carboxyl group is amidated or esterified. The ester used in this case includes e.g. the ester at the C-terminal as described above.

Also, the protein of the invention includes those proteins wherein an amino group of an N-terminal amino acid residue (e.g. methionine residue) is protected with a protective group (e.g., C₁₋₆ acyl group including C₁₋₆ alkanoyl such as formyl group and acetyl group), an N-terminal glutamic residue formed in vivo by cleavage is oxidized into pyroglutamine, or a substituent (e.g., —OH, —SH, amino group, imidazole group, indole group, guanidino group or the like) on a side chain of an intramolecular amino acid is protected with a suitable protective group (e.g., C₁₋₆ acyl group including C₁₋₆ alkanoyl such as formyl group and acetyl group), as well as conjugated proteins such as glycoprotein having sugar chains bound thereto.

As the protein of the invention, for example a human-derived (preferably human kidney-derived) protein having the amino acid sequence set forth in SEQ ID NO:4 is used.

The partial peptide of the protein of the invention may be any partial peptide which is derived from the protein of the invention described above and preferably has similar properties (for example, the activity of binding to NKG2D, the action of activating immunocytes etc.) to those of the protein of the invention described above. As the partial peptide of the invention, use is made of peptides having at least 20%, preferably 50% or more, more preferably 70% or more, still more preferably 90% or more, and most preferably 95% or more of the amino acid sequence constituting the protein of the invention and having the activity of binding to NKG2D or the action of activating immunocytes.

Also, the partial peptide of the invention may have deletion of 1 to 5 (preferably 1 to 3) amino acids in the amino acid sequence, addition of 1 to 10 (preferably 1 to 5 (more preferably 1 to 3)) amino acids in the amino acid sequence, insertion of 1 to 5 (preferably 1 to 3) amino acids in the amino acid sequence, or replacement of 1 to 5 (preferably 1 to 3) amino acids by other amino acids in the amino acid sequence.

Specifically, the partial peptide of the invention includes a partial peptide having a partial amino acid sequence between positions 24 to 255 in the amino acid sequence shown in SEQ ID NO:4, a partial peptide having a partial amino acid sequence between positions 31 to 255 in the amino acid sequence shown in SEQ ID NO:4, a partial peptide having a partial amino acid sequence between positions 26 to 255 in the amino acid sequence shown in SEQ ID NO:4, a partial peptide having a partial amino acid sequence between positions 25 to 255 in the amino acid sequence shown in SEQ ID NO:4, and a partial peptide having a partial amino acid sequence between positions 27 to 255 in the amino acid sequence shown in SEQ ID NO:4.

The partial peptide of the invention usually has a carboxyl group (—COOH) or carboxylate (—COO—) at the C-terminal, but like the protein of the invention described above, the partial peptide may have an amide (—CONH) or ester (—COOR) (R has the same meaning as defined above) at the C-terminal.

Like the protein of the invention described above, the partial peptide of the invention includes those peptides wherein an amino group of an N-terminal amino acid residue (e.g. methionine residue) is protected with a protective group, an N-terminal glutamine residue is formed in vivo by cleavage of the N-terminal and converted into pyroglutamic acid, or a substituent on a side chain of an intramolecular amino acid is protected with a suitable protective group, as well as conjugated peptides such as glycopeptide having sugar chains bound thereto.

Because the partial peptide of the invention can be used as an antigen for raising an antibody, it is not always necessary that the partial peptide has the activity of binding to NKG2D or the action of activating immunocytes.

The protein of the invention, its partial peptide, or DNA coding for the protein of the invention or its partial peptide may be labeled in a method known per se, and includes e.g. those labeled with an isotope, with a fluorescent label (for example, a fluorescent label such as fluorescein), with biotin, with an enzyme etc.

As the salts of the protein or partial peptide of the invention, salts with physiologically acceptable acids (e.g., inorganic acids, organic acids) or bases (e.g., alkali metal salts) are used, and in particular physiologically acceptable acid-addition salts are preferable. Such salts include e.g. salts with inorganic acids (e.g., hydrochloric acid, phosphoric acid, hydrobromic acid, sulfuric acid) or salts with organic acids (e.g., acetic acid, formic acid, propionic acid, fumaric acid, maleic acid, succinic acid, tartaric acid, citric acid, malic acid, oxalic acid, benzoic acid, methanesulfonic acid, benzenesulfonic acid).

The protein or partial peptide of the invention or salts thereof can be produced by known protein purification techniques from the human or warm-blooded animal cells or tissues (particularly kidney etc.) described above, or by culturing transformants containing DNA coding for the protein as described later. Alternatively, these can also be produced by a peptide synthesis method described below.

For production from human or mammalian tissues or cells (particularly kidney etc.), the human or mammalian tissues or cells are homogenized and then extracted with e.g. an acid, and the extract can be purified and isolated by a combination of chromatographic techniques such as reverse-phase chromatography, ion-exchange chromatography etc.

For production from human or mammalian tissues or cells (particularly kidney etc.), the human or mammalian tissues or cells are homogenized and then extracted with e.g. an acid, and the extract can be purified and isolated by a combination of chromatographic techniques such as reverse-phase chromatography, ion-exchange chromatography etc.

For synthesis of the protein or partial peptide of the invention or salts thereof or amide derivatives thereof, usually commercially available resin for protein synthesis can be used. Such resin includes e.g. chloromethyl resin, hydroxymethyl resin, benzhydryl amine resin, aminomethyl resin, 4-benzyloxybenzyl alcohol resin, 4-methylbenzhydryl amine resin, PAM resin, 4-hydroxymethyl methyl phenyl acetamide methyl resin, polyacrylamide resin, 4-(2′,4′-dimethoxyphenyl-hydroxymethyl) phenoxy resin, 4-(2′,4′-dimethoxyphenyl-Fmoc aminoethyl) phenoxy resin etc. On the resin described above, each amino acid whose α-amino group and side-chain functional group are properly protected is condensed sequentially in accordance with the sequence of the desired protein by various condensation methods known per se. At the end of the reaction, the protein is cleaved off from the resin while various protective groups are removed, and the product is subjected to an intramolecular disulfide bond-forming reaction in a highly diluted solution to give the desired protein or an amide thereof.

Although a wide variety of activating reagents usable for protein synthesis can be used for condensation of the protected amino acids described above, carbodiimides are particularly preferable. Examples of such carbodiimides include DCC, N,N′-diisopropylcarbodiimide, N-ethyl-N′-(3-dimethylaminoprolyl) carbodiimide, etc. For activation by these reagents, the protected amino acids along with racemization inhibitors (e.g., HOBt, HOOBt) can be added to the resin directly or after the protected amino acids are previously activated as symmetric acid anhydrides or HOBt esters or HOOBt esters.

The solvent used for activation of each protected amino acid or for condensation thereof with the resin can be selected as necessary from those solvents known to be usable in protein condensation reaction. Examples of such solvent include acid amides such as N,N-dimethylformamide, N,N-dimethylacetamide and N-methylpyrrolidone, hydrocarbon halides such as methylene chloride and chloroform, alcohols such as trifluoroethanol, sulfoxides such as dimethyl sulfoxide, ethers such as pyridine, dioxane and tetrahydrofuran, nitriles such as acetonitrile and propionitrile, esters such as methyl acetate and ethyl acetate, or a suitable mixture thereof. The reaction temperature is usually selected as necessary within the range known to be usable in the reaction of forming protein bonds, and usually the reaction temperature is selected within the range of −20° C. to 50° C. The activated amino acid derivatives are used usually in excess (1.5- to 4-fold). When the condensation is insufficient as a result of a test using ninhydrin reaction, the condensation reaction is repeatedly carried out without conducting elimination of the protective groups, whereby sufficient condensation can be achieved. When sufficient condensation cannot be achieved even by repeatedly carrying out the reaction, the unreacted amino acids are acetylated with acetic anhydride or acetyl imidazole so that the subsequent reaction cannot be influenced.

The protective groups for amino groups in the raw materials include e.g. Z, Boc, t-pentyloxycarbonyl, isobornyloxycarbonyl, 4-methoxybenzyloxycarbonyl, Cl—Z, Br—Z, adamantyloxycarbonyl, trifluoroacetyl, phthaloyl, formyl, 2-nitrophenylsulphenyl, diphenylphospinothioyl, Fmoc etc.

The carboxyl group can be protected by e.g. alkyl esterification (e.g., linear, branched or cyclic alkyl esterification such as methyl, ethyl, propyl, butyl, t-butyl, cyclopentyl, cyclohexyl, cycloheptyl, cycloctyl or 2-adamantyl esterification), aralkyl esterification (e.g., benzyl esterification, 4-nitrobenzyl esterification, 4-methoxybenzyl esterification, 4-chlorobenzyl esterification, benzhydryl esterification), phenacyl esterification, benzyloxy carbonyl hydrazidation, t-butoxycarbonyl hydrazidation, trityl hydrazidation etc.

The hydroxyl group in serine can be protected by e.g. esterification or etherification. A suitable group used in this esterification includes e.g. lower (C₁₋₆) alkanoyl groups such as acetyl group, aroyl groups such as benzoyl group, and carbonic acid-derived groups such as benzyloxycarbonyl group and ethoxycarbonyl group. A suitable group for etherification includes e.g. a benzyl group, tetrahydropyranyl group, t-butyl group etc.

The protective group used for the phenolic hydroxyl group in tyrosine includes e.g. Bzl, Cl₂-Bzl, 2-nitrobenzyl, Br—Z, t-butyl etc.

The protective group used for imidazole in histidine includes e.g. Tos, 4-methoxy-2,3,6-trimethylbenzene sulfonyl, DNP, benzyloxymethyl, Bum, Boc, Trt, Fmoc etc.

The raw materials having activated carboxyl groups include e.g. the corresponding acid anhydrides, azides and active esters (i.e. esters with alcohols such as pentachlorophenol, 2,4,5-trichlorophenol, 2,4-dinitrophenol, cyanomethyl alcohol, p-nitrophenol, HONB, N-hydroxysuccimide, N-hydroxyphthalimide and HOBt). The raw materials having activated amino groups include e.g. the corresponding phosphoric acid amides.

The method for removal (elimination) of the protective groups makes use of e.g. catalytic reduction in a hydrogen stream in the presence of a catalyst such as Pd-black or Pd-carbon, acid treatment with anhydrous hydrogen fluoride, methane sulfonic acid, trifluoromethane sulfonic acid, trifluoroacetic acid or a mixed solution thereof, base treatment with diisopropylethylamine, triethylamine, piperidine or piperazine, and reduction with sodium in liquid ammonia. The elimination reaction by the acid treatment is carried out generally at a temperature of about −20° C. to 40° C., and in the acid treatment, addition of cation scavengers such as anisole, phenol, thioanisole, m-cresol, p-cresol, dimethyl sulfide, 1,4-butane dithiol and 1,2-ethane dithiol is effective. A 2,4-dinitrophenyl group used as a protective group for imidazole in histidine can also be removed by treatment with thiophenol, while a formyl group used as a protective group for indole in tryptophan can be removed not only by deprotection by acid treatment in the presence of 1,2-ethane dithiol or 1,4-butane dithiol, but also by alkali treatment with a diluted sodium hydroxide solution or diluted ammonia.

Protection and protective groups for functional groups which should not participate in the reaction of the raw materials, elimination of the protective groups, and activation of functional groups participating in the reaction can be selected as necessary from known groups or known means.

Another method of obtaining an amide derivative of the protein involves protecting the a-carboxyl group of a C-terminal amino acid by amidation, then extending a peptide (protein) chain at the side of the amino group until it attains desired chain length, and thereafter producing a protein of said peptide chain from which only the protective group for the N-terminal α-amino group has been removed and a protein of said peptide chain from which only the protective group for the C-terminal carboxyl group has been removed, followed by condensation of both the proteins in the mixed solvent described above. The details of the condensation reaction are the same as described above. The protected protein obtained by condensation is purified, and every protective group is removed by the method descried above, whereby the desired crude protein can be obtained. This crude protein is purified by a wide variety of known purification techniques, and by lyophilizing its main fraction, the desired amide derivative of the protein can be obtained.

To obtain an ester derivative of the protein, for example the α-carboxyl group of a C-terminal amino acid is condensed with desired alcohol to form an amino acid ester from which the desired ester derivative of the protein can be obtained in the same manner as for the amide derivative of the protein.

The partial peptide of the invention or salts thereof can be produced according to a peptide synthesis method known per se or by cleaving the protein of the invention with a suitable peptidase. For example, the peptide synthesis method may be the solid- or liquid-phase synthesis method. That is, the desired peptide can be obtained by condensation of a partial peptide or amino acids capable of constituting the partial peptide of the invention with the remainder, followed by elimination of protective groups if any from the product. As the known condensation method and the elimination of the protective groups, mention is made of e.g. the methods described in (1) to (5) below:
(1) M. Bodanszky and M. A. Ondetti, Peptide Synthesis, Interscience Publishers, New York (1966);
(2) Schroeder and Luebke, The Peptide, Academic Press, New York (1965);
(3) N. Izumiya et al., Basis and Experiments in Peptide Synthesis (in Japanese), Maruzen K. K. (1975);
(4) T. Yajima & S. Sakakibara, Biochemical Experimental Lecture 1, Protein Chemistry IV, 205 (1977) (in Japanese); and
(5) A Sequel to Development of Pharmaceutical Preparations, Vol. 14, Peptide Synthesis (in Japanese), supervised by T. Yajima, Hirokawa Shoten.

Further, after the reaction, the partial peptide of the invention can be isolated and purified by a combination of conventional purification techniques such as solvent extraction, distillation, column chromatography, liquid chromatography and recrystallization. If the partial peptide is obtained in a free form by these methods, the product can be converted into a suitable salt by a known method or its analogous method, or if the partial peptide is obtained in a salt form, it can be converted into a free peptide or other salts by a known method or its analogous method.

The DNA coding for the protein of the invention may be any DNA containing a nucleotide sequence coding for the protein of the invention described above. This DNA may also be genomic DNA, a genomic DNA library, cDNA derived form the above-described cells or tissues, a cDNA library derived form the above-described cells or tissues, or synthetic DNA.

The vector used in the library may be bacteriophage, plasmid, cosmid, phagimide or the like. A total RNA or mRNA fraction prepared from the cells and tissues described above can also be used in direct amplification by Reverse Transcriptase Polymerase Chain Reaction (abbreviated hereinafter to RT-PCR method).

The DNA coding for the protein of the invention includes e.g. DNA containing DNA having the nucleotide sequence set forth in SEQ ID NO:3, or DNA having a nucleotide sequence hybridizing under high stringent conditions with the nucleotide sequence set forth in SEQ ID NO:3 and coding for a protein having substantially identical properties (e.g., the activity of binding to NKG2D, the action of activating immunocytes, etc.) with those of the protein of the invention.

The high stringent conditions refer to those conditions under which the concentration of sodium is about 19 to 40 mM, preferably about 19 to 20 mM, and the temperature is about 50 to 70° C., preferably about 60 to 65° C. In particular, the high stringent conditions are preferably those conditions under which the concentration of sodium is about 19 mM, and the temperature is about 65° C.

Specifically, the DNA coding for a protein having the amino acid sequence set forth in SEQ ID NO:4 includes DNA having the nucleotide sequence set forth in SEQ ID NO:3.

The DNA coding for the partial peptide of the invention may be any DNA containing a nucleotide sequence encoding the above-described partial peptide of the invention. It may also be genomic DNA, a genomic DNA library, cDNA derived form the above-described cells or tissues, a cDNA library derived form the above-described cells or tissues, or synthetic DNA.

The DNA coding for the partial peptide of the invention includes e.g. DNA having a partial nucleotide sequence of DNA having the nucleotide sequence set forth in SEQ ID NO:3, or DNA having a nucleotide sequence hybridizing under high stringent conditions with the nucleotide sequence set forth in SEQ ID NO:3 and having a partial nucleotide sequence of DNA coding for a protein having substantially identical properties with those of the protein of the invention.

As a means of cloning the DNA coding completely for the protein and partial peptide of the invention (these proteins are referred to collectively as the protein of the invention in the following description of cloning and expression of the DNA coding for these proteins), it is possible to use amplification by the known PCR method using synthetic DNA primers having a partial nucleotide sequence of the protein of the invention, or selection by hybridization of the DNA integrated in a suitable vector with a labeled DNA fragment or synthetic DNA coding for a part or the whole of the protein of the invention. Hybridization can be carried out according to a method described in e.g. Molecular Cloning, 2nd ed., J. Sambrook et al., Cold Spring Harbor Lab. Press, 1989. When a commercial library is used, hybridization can be carried out according to its accompanying protocol.

Conversion of the nucleotide sequence of DNA can be carried out by a method known per se, for example by the Gupped duplex method or Kunkel method or its analogous method by using a known kit such as Mutan™-G (Takara Shuzo K. K.) or Mutan T-K (Takara Shuzo K. K.).

The cloned DNA coding for the protein may be used as such depending on the object or if necessary after digestion with restriction enzymes or after addition of a linker. The DNA may have ATG as a translation initiation codon at the 5′-terminal thereof and TAA, TGA or TAG as a translation termination codon at the 3′-terminal thereof These translation initiation and termination codons can also be added to the DNA via a suitable synthetic DNA adaptor.

An expression vector for the protein of the invention can be produced for example by (A) cutting a desired DNA fragment off from the DNA coding for the protein of the invention and then (B) ligating the DNA fragment to a region downstream from a promoter in a suitable expression vector.

The vector used includes E. coli-derived plasmids (e.g., pBR322, pBR325, pUC12, pUC13), Bacillus subtilis-derived plasmids (e.g., pUB110, pTP5, pC194), yeast-derived plasmids (e.g., pSH19, pSH15), bacteriophage such as λ-phage, and animal viruses such as retrovirus, vaccinia virus and baculovirus, as well as pA1-11, pXT1, pRc/CMV, pRc/RSV, pcDNAI/Neo etc.

The promoter used in this invention may be any suitable promoter compatible with a host used for expression of the gene. For example, when animal cells are used as the host, mention is made of SRα promoter, SV40 early promoter, HIV-LTR promoter, CMV promoter, HSV-TK promoter etc.

Among these promoters, CMV (cytomegalovirus) promoter, SRα promoter etc. are preferably used. It is preferable to use trp promoter, lac promoter, recA promoter, λPL promoter, lpp promoter, T7 promoter etc. for microorganisms of the genus Escherichia as the host, SPO1 promoter, SPO2 promoter, penP promoter etc. for microorganisms of the genus Bacillus as the host, and PHO5 promoter, PGK promoter, GAP promoter, ADH promoter etc. for yeasts as the host. When insect cells are used as the host, polyhedron promoter, P10 promoter etc. are preferable.

The expression vector may contain an enhancer, a splicing signal, a poly A-added signal, a selective marker, an SV40 origin of replication (also referred to hereinafter as SV40 ori) etc. if necessary in addition to the element described above. The selective marker includes e.g. dihydrofolate reductase (also referred to hereinafter as dhfr) gene [methotrexate (MTX) resistance], ampicillin resistance gene (also referred to hereinafter as Amp^{r}) and neomycin resistance gene (G418 resistance, also referred to hereinafter as Neo^{r}). In particular, when the dhfr gene is used as a selective marker for dhfr gene-defective Chinese hamster cells, the desired gene can also be selected in a thymidine-free medium.

A signal sequence compatible with the host is added if necessary to the sequence corresponding to the N-terminal of the protein of the invention. A PhoA signal sequence, Omp A signal sequence etc. can be utilized for microorganisms of the genus Escherichia used as the host; an α-amylase signal sequence, subtilisin signal sequence etc. for microorganisms of the genus Bacillus as the host; an MFα signal sequence, SUC2 signal sequence etc. for yeasts as the host; and an insulin signal sequence, α-interferon signal sequence, antibody molecule signal sequence etc. for animal cells as the host.

The thus constructed vector containing the DNA coding for the protein of the invention can be used to produce transformants.

Microorganisms of the genus Escherichia, microorganisms of the genus Bacillus, yeasts, insect cells, insects, animal cells etc. are used as the host.

The microorganisms of the genus Escherichia used include e.g. Escherichia coli K12 DH1 (Proc. Natl. Acad. Sci. USA, vol. 60, 160 (1968)), JM103 (Nucleic Acids Research, vol. 9, 309 (1981)), JA221 (Journal of Molecular Biology, vol. 120, 517 (1978)), HB101 (Journal of Molecular Biology, vol. 41, 459 (1969)), C600 (Genetics, vol. 39, 440 (1954)), etc.

The microorganisms of the genus Bacillus used include e.g. Bacillus subtilis MI114 (Gene, vol. 24, 255 (1983)), 207-21 (Journal of Biochemistry, vol. 95, 87 (1984)), etc.

The yeasts used include Saccharomyces cerevisiae AH22, AH22R⁻, NA87-l1A, DKD-5D, 20B-12, Schizosaccharomyces pombe NCYC1913, NCYC2036, Pichia pastoris KM71, etc.

For example, when the virus is AcNPV, the insect cells used include e.g. cells (Spodoptera frugiperda cells; Sf cells) from an established cell line derived from caterpillars of Spodoptera frugiperda, MG1 cells derived from the midgut in Trichoplusia ni, High Five™ cells derived from eggs of Trichoplusia ni, cells derived from Mamestra brassicae and cells derived from Estigmena acrea. When the virus is BmNPV, cells (Bombyx mori N cells; BmN cells) from an established cell line derived from silkworms, etc., are used. The Sf cells used include e.g. Sf9 cells (ATCC CRL1711), Sf21 cells (Vaughn, J. L. et al., In Vivo, 13, 213-217 (1977)), etc.

The insects used include e.g. silkworm caterpillars (Maeda et al., Nature, vol. 315, 592 (1985)).

The animals cells used include e.g. simian cell COS-7 (COS-7), Vero, Chinese hamster ovary cells CHO (abbreviated hereinafter to CHO cells), dhfr gene-defective Chinese hamster cells CHO (abbreviated hereinafter to CHO (dhfr⁻) cells), mouse L cells, mouse AtT-20, mouse myeloma cells, rat GH3, human FL cells etc. Further, use can also be made of various normal human cells such as hepatic cells, spleen cells, nerve cells, glia cells, pancreatic β-cells, bone marrow cells, mesangial cells, Langerhans cells, epidermal cells, epithelial cells, endothelial cells, fibroblasts, fibrous cells, muscular cells, adipocytes, immunocytes (e.g., macrophage, T cells, B cells, natural killer cells, mast cells, neutrophils, basophils, acidophils, monocytes), megakaryocytes, synovial membrane cells, cartilage cells, bone cells, osteoblasts, osteoclasts, mammary gland cells, hepatic cells or interstitial cells, or their precursor cells, stem cells or cancer cells).

The microorganisms of the genus Escherichia can be transformed according to a method described in e.g. Proc. Natl. Acad. Sci. USA, vol. 69, 2110 (1972) or Gene, vol. 17, 107 (1982).

The microorganisms of the genus Bacillus can be transformed according to a method described in e.g. Molecular & General Genetics, vol. 168, 111 (1979), etc.

The yeasts can be transformed according to a method described in e.g. Methods in Enzymology, vol. 194, 182-187 (1991), Proc. Natl. Acad. Sci. USA, vol. 75, 1929 (1978), etc.

The insect cells or insects can be transformed according to a method described in e.g. Bio/Technology, 6, 47-55 (1988), etc.

The animal cells can be transformed according to a method described in e.g. Cell Technology, Extra Number 8, New Experimental Protocol in Cell Technology”, 263-267 (1995) (published by Shujunsha), Virology, vol. 52, 456 (1973), etc.

Transformants transformed with the expression vector containing DNA coding for the protein can be thus obtained.

When transformants derived from the microorganisms of the genus Escherichia or Bacillus as the host are cultured, the medium used for their culture is preferably a liquid medium containing a carbon source, a nitrogen source, inorganic matter etc. necessary for growth of the transformants. The carbon source includes e.g. glucose, dextrin, soluble starch, sucrose etc.; the nitrogen source includes e.g. inorganic or organic materials such as ammonium salts, nitrates, corn steep liquor, peptone, casein, meat extract, soybean cake and potato extract; and the inorganic matter includes e.g. calcium chloride, sodium dihydrogen phosphate, magnesium chloride etc. In addition, a yeast extract, vitamins, growth promoting factors etc. may be added. The pH value of the medium is desirably about 5 to 8.

For example, the medium for culturing the microorganisms of the genus Escherichia is preferably M9 medium containing glucose and casamino acid (Miller, Journal of Experiments in Molecular Genetics, 431-433, Cold Spring Harbor Laboratory, New York, 1972). To permit the promoter to work efficiently if necessary, a chemical such as 3β-indolylacrylic acid can be added thereto.

The transformants from the microorganisms of the genus Escherichia as the host are cultured usually at about 15 to 43° C. for about 3 to 24 hours during which the medium may be aerated or stirred as necessary.

The transformants from the microorganisms of the genus Bacillus as the host are cultured usually at about 30 to 40° C. for about 6 to 24 hours during which the medium may be aerated or stirred as necessary.

The medium used for culturing the transformants from yeasts as the host includes e.g. Burkholder minimum medium (Bostian, K. L. et al., Proc. Natl. Acad. Sci. USA, vol. 77, 4505 (1980)) and SD medium containing 0.5% casamino acid (Bitter, G. A. et al., Proc. Natl. Acad. Sci. USA, vol. 81, 5330 (1984)). The pH value of the medium is adjusted preferably to about 5-8. The transformants are cultured usually at about 20 to 35° C. for about 24 to 72 hours during which the medium may be aerated or stirred as necessary.

The medium used for culturing the transformants from insect cells or insects as the host includes e. g. a medium prepared by adding inactivated additives such as 10% bovine serum to Grace's insect medium (Grace, T. C. C., Nature, 195, 788 (1962)). The pH value of the medium is adjusted preferably to about 6.2-6.4. The transformants are cultured usually at about 27° C. for about 3 to 5 days during which the medium may be aerated or stirred as necessary.

The medium used for culturing the transformants from animal cells as the host includes e.g. MEM medium containing about 5 to 20% FBS (Science, 122, 501 (1952)), DMEM medium (Virology, vol. 8, 396 (1959)), RPMI 1640 medium (The Journal of the American Medical Association, vol. 199, 519 (1967)), 199 medium (Proceeding of the Society for the Biological Medicine, vol. 73, 1 (1950)) etc. The pH value is preferably about 6 to 8. The transformants are cultured usually at about 30 to 40° C. for about 15 to 60 hours during which the medium may be aerated or stirred as necessary.

Thus, the protein of the invention can be formed in the cells or out of the cells of the transformants.

From the resulting culture, the protein of the invention can be separated and purified for example in the following manner.

To extract the protein of the invention from the cultured microorganisms or cells, the cultured microorganisms or cells are collected in a usual manner, suspended in a suitable buffer, disrupted by sonication, lysozyme and/or freezing and thawing, and centrifuged or filtered to give a crude extract of the protein. The buffer may contain protein denaturants such as urea and guanidine hydrochloride and surfactants such as Triton X-100™. When the protein is secreted into the culture liquid, the culture supernatant is collected by separating the supernatant from the cultured microorganisms or cells by a method known per se.

The culture supernatant thus obtained, or the protein contained in the extract, can be purified by a suitable combination of separation and purification techniques known per se. These known separation and purification techniques make use of a method of utilizing solubility, such as salting-out and solvent precipitation, a method of mainly utilizing a difference in molecular weight, such as dialysis, ultrafiltration, gel filtration, and SDS-polyacrylamide gel electrophoresis, a method of utilizing a difference in electric charge, such as ion-exchange chromatography, a method of utilizing specific affinity, such as affinity chromatography, a method of utilizing a difference in hydrophobicity, such as reverse-phase HPLC, a method of utilizing a difference in isoelectric point, such as isoelectric focusing.

If the protein thus obtained is in a free form, it can be converted into a salt by a method known per se or its analogous method, while if the resulting protein is obtained in the form of a salt, it can be converted into a free protein or another salt by a method known per se or its analogous method.

Before or after purification, a suitable protein-modifying enzyme may be act on act on the protein produced by the transformants, whereby the protein can be arbitrarily modified or a partial polypeptide can be removed therefrom. For example, trypsin, chymotrypsin, arginyl endopeptidase, protein kinase or glycosidase is used as the protein-modifying enzyme.

The presence or activity of the thus formed protein of the invention or salts thereof can be measured by an experiment of binding with a labeled ligand and enzyme immunoassays with specific antibody.

The antibody against the protein and partial peptide of the invention or salts thereof may be a polyclonal or monoclonal antibody capable of recognizing the protein or partial peptide of the invention or salts thereof The antibody against the protein and partial peptide of the invention or salts thereof (referred to collectively as the protein of the invention in the following description of the antibody) can be produced by a known process for producing antibody or antiserum by using the protein of the invention as the antigen.

[Preparation of the Monoclonal Antibody]

(a) Preparation of Monoclonal Antibody-Producing Cells

The protein of the invention is administered alone or together with a carrier and a diluent into warm-blooded animals at a site where the antibody can be produced by administration. To enhance the ability of the animals upon administration to produce the antibody, complete Freund's adjuvant or incomplete Freund's adjuvant may be administered. Administration is conducted usually once every 2 to 6 weeks and about 2 to 10 times in total. The warm-blooded animals used include e.g. monkey, rabbit, dog, guinea pig, mouse, rat, sheep, goat and chicken, among which mouse and rat are preferably used.

For production of the monoclonal antibody-producing cells, those animals having antibody titer are selected from the warm-blooded animals (e.g. mice) immunized with the antigen, and on the second to fifth day after the final immunization, their spleens or lymph nodes are collected, and the antibody-producing cells contained therein are fused with myeloma cells from animals of the same or different species, whereby monoclonal antibody-producing hybridomas can be produced. The antibody titer in antiserum can be measured for example by reacting the antiserum with a labeled protein described later and then measuring the activity of a label bound to the antibody. Fusion can be carried out by a known method such as the method of Kohler and Millstein (Nature, 256, 495 (1975)). The fusion promoter includes polyethylene glycol (PEG) and Sendai virus, and PEG is preferably used.

The myeloma cells include myeloma cells NS-1, P3U1, SP2/0 and AP-1 from warm-blooded animals, among which P3U1 is preferably used. The ratio of the antibody-producing cells (spleen cells) to the myeloma cells used is from about 1:1 to 20:1, and cell fusion can be effected efficiently by incubating the cells for about 1 to 10 minutes at 20 to 40° C., preferably 30 to 37° C., in the presence of PEG (preferably PEG 1000 to PEG 6000) at a concentration of about 10 to 80%.

The monoclonal antibody-producing hybridoma can be screened by various methods, for example by adding a culture supernatant of the hybridoma to a solid phase (e.g., a microplate) having the protein antibody adsorbed thereon directly or along with a carrier and then adding a radioactive substance- or enzyme-labeled anti-immunoglobulin antibody (which is e.g. an anti-mouse immunoglobulin antibody when mouse cells are subjected to cell fusion) or protein A to detect the monoclonal antibody bound to the solid phase or by adding a culture supernatant of the hybridoma to a solid phase having an anti-immunoglobulin antibody or protein A adsorbed thereon and then adding the protein labeled with a radioactive substance or an enzyme to detect the monoclonal antibody bound to the solid phase.

The monoclonal antibody can be screened in a method known per se or its analogous method. Screening can be carried out usually in an animal cell culture medium to which HAT (hypoxanthine, aminopterin, thymidine) was added. The screening and breeding medium may be any medium in which the hybridoma can grow. Examples of such medium include PRMI 1640 medium containing 1 to 20% (preferably 10 to 20%) FBS, GIT medium containing 1 to 10% FBS (Wako Pure Chemical Industries, Ltd.), and a serum-free medium for hybridoma culture (SFM-101, Nissui Seiyaku K. K.). The culture temperature is usually 20 to 40° C., preferably about 37° C. The culture time is usually 5 days to 3 weeks, preferably 1 to 2 weeks. Culture can be conducted usually in 5% CO₂ gas. The antibody titer in a culture supernatant of the hybridoma can be measured in the same manner as in the measurement of the antibody titer in antiserum as described above.

(b) Purification of the Monoclonal Antibody

The monoclonal antibody can be separated and purified by techniques known per se, for example by techniques of separating and purifying immunoglobulins, such as salting-out, alcohol precipitation, isoelectric precipitation, electrophoresis, absorption-desorption with an ion-exchanger (e.g. DEAE), ultracentrifugation and gel filtration or by specific purification of the antibody by collecting it with an active adsorbent such as antigen-bound solid phase, protein A or protein G and then eluting the antibody therefrom.

[Preparation of the Polyclonal Antibody]

The polyclonal antibody of the invention can be produced by a method known per se or its analogous method. For example, the desired polyclonal antibody can be produced by preparing the immune antigen (protein antigen) or a conjugate thereof with a carrier protein, then immunizing warm-blooded animals therewith in the same manner as in production of the monoclonal antibody as described above, collecting a material containing the antibody against the protein of the invention from the immunized animals, and separating and purifying the antibody.

For the conjugate of the immune antigen with a carrier protein used for immunizing warm-blooded animals, the type of the carrier protein and the mixing ratio of the carrier to the hapten are not particularly limited insofar as the desired antibody can be efficiently produced by immunization with the hapten crosslinked with the carrier, and for example, the conjugate is produced by coupling the hapten with bovine serum albumin, bovine cyloglobulin, hemocyanin or the like in a ratio of about 0.1-20:1, preferably about 1-5:1.

The hapten can be coupled with the carrier by use of various condensation agents such as glutaraldehyde, carbodiimide, maleimide-activated ester, and activated ester reagents containing thiol group and dithiopyridyl group.

The resulting condensation product is administered alone or together with a carrier and a diluent into warm-blooded animals at a site where the antibody can be produced. To enhance the ability of the animals upon administration to produce the antibody, complete Freund's adjuvant or incomplete Freund's adjuvant may be administered. Administration is conducted usually once every 2 to 6 weeks and about 3 to 10 times in total.

The polyclonal antibody can be collected form blood, ascites etc. preferably blood in the warm-blooded animals immunized by the method described above.

The polyclonal antibody titer in antiserum can be measured in the same manner as in the measurement of the antibody titer in antiserum as described above. Separation and purification of the polyclonal antibody can be carried out by a method of separating and purifying immunoglobulins, which is similar to the separation and purification of the monoclonal antibody as described above.

Hereinafter, use of the protein or partial peptide of the invention or salts thereof (also referred to hereinafter as the protein etc. of the invention) and the antibody against the protein or partial peptide of the invention or against salts thereof (also referred to hereinafter as the antibody of the invention) is described.

(1) An Agent for Treating and Preventing Various Diseases, Which Contains the Protein etc. of the Invention

The protein etc. of the invention have the activity of binding to NKG2D which is a receptor observed to be expressed in immunocytes etc. NKG2D is expressed in mainly NK cells and also in a part of T cells, and known as a receptor activating these cells. Accordingly, the protein etc. of the invention can be expected to activate immunocytes via NKG2D. The protein etc. of the invention can be used in therapy and prevention of various diseases thorough its immune activating activity. That is, the protein etc. of the invention can be used not only in an agent for treating and preventing diseases caused by infection with microorganisms, new microorganisms and viruses but also in an agent for treating and preventing diseases such as various cancers (for example, uterine cancer, endometrium tumor, breast cancer, colon cancer, prostate cancer, lung cancer, kidney cancer, neuroblastoma, bladder cancer, melanoma etc.). In particular, the protein etc. of the invention is useful as a medicine for prevention of recurrence and complete treatment of various cancers after excision of carcinoma by its immune activating action.

When the protein etc. of the invention are used as the therapeutic or preventive agent described above, the protein etc. of the invention are purified to a purity of at least 90%, preferably 95% or more, more preferably 98% or more, still more preferably 99% or more.

For example, the protein etc. of the invention can be used orally as tablets coated with sugar as necessary, capsules, elixirs and microcapsules or parenterally as an aseptic solution with water or with other pharmaceutically acceptable solutions or as an injection such as suspension. For example, these compositions can be produced by admixing physiologically acceptable carriers, flavorings, excipients, vehicles, preservatives, stabilizers and binders with the protein etc. of the invention in a unit dose required for generally approved pharmaceutical manufacturing. The amount of the active ingredient in these pharmaceutical compositions is designed to have a suitable capacity in the designated range.

The additives which can be admixed with the tablets, capsules etc. include e.g. binders such as gelatin, corn starch, tragacanth, Arabia gum, excipients such as crystalline cellulose, swelling agents such as corn starch, gelatin and alginic acid, lubricants such as magnesium stearate, sweeteners such as sucrose, lactose and saccharine, and flavorings such as peppermint, Gaultheria adenothrix oil and cherry. When one capsule is in a unit form, liquid carriers such as fats and oils can be contained in the materials described above. The aseptic composition for injection can be formulated according to conventional pharmaceutical manufacturing by dissolving or suspending the active material and naturally occurring vegetable oils such as sesame oil and coconut oil in vehicles such as injection water.

The aqueous solution for injection includes e.g. physiological saline or an isotonic solution containing glucose and other auxiliary chemicals (e.g., D-sorbitol, D-mannitol, sodium chloride etc.), and may be used in combination with suitable solubilizers such as alcohols (e.g., ethanol etc.), polyalcohols (e.g., propylene glycol, polyethylene glycol etc.) and nonionic surfactants (e.g., Polysorbate 80™, HCO-50 etc.). The oily solution includes e.g. sesame oil, soybean oil etc., and may be used in combination with solubilizers such as benzyl benzoate, benzyl alcohol etc. Further, it may be compounded with the buffer (e.g., phosphate buffer, sodium acetate buffer etc.), analgesic agents (e.g., benzalkonium chloride, procaine hydrochloride etc.), stabilizers (e.g., human serum albumin, polyethylene glycol etc.), preservatives (e.g., benzyl alcohol, phenol etc.), antioxidants etc. Usually, the prepared injection is introduced into suitable ampoules.

The pharmaceutical composition thus obtained is safe and low toxic so that it can be administered into e.g. humans or warm-blooded animals (e.g., rat, mouse, guinea pig, rabbit, bird, sheep, pig, cow, horse, cat, dog, monkey etc.).

The dose of the protein etc. of the invention is varied depending on the intended disease, the subject of administration etc., and when the protein etc. of the invention are administered for example as an anticancer drug, the protein etc. are administered usually in a dose of about 0.1 to 100 mg, preferably about 1.0 to 50 mg, more preferably about 1.0 to 20 mg every 10 day into a (60 kg) adult.

(2) Screening of Candidates for Pharmaceutical Compounds Against Diseases

The compound or a salt thereof inhibiting the activity of the protein of the invention, that is, an antagonist for the protein of the invention, can be expected to suppress immunocytes. This antagonist can be used as an immunosupressant or an antiinflammatory agent not only against various diseases such as autoimmune diseases and infectious hyper-immunoreactions, but also for suppression of immunoreactions after transplantation of organs and tissues.

Like the protein of the invention, the compound or a salt thereof promoting the activity of the protein of the invention, that is, an agonist for the protein of the invention, can be used for treating and preventing various diseases by its immune activating action. That is, it can be used in pharmaceutical compositions including not only an agent for treating and preventing diseases caused by infection with microorganisms, new microorganisms and viruses but also an agent for treating and preventing diseases such as various cancers (for example, uterine cancer, endometrium tumor, breast cancer, colon cancer, prostate cancer, lung cancer, kidney cancer, neuroblastoma, bladder cancer, melanoma etc.).

Accordingly, the protein etc. of the invention are useful as a reagent for screening a compound or a salt thereof having the activity of promoting or inhibiting the binding of the protein etc. of the invention to NKG2D or the activation of immunocytes.

That is, this invention provides:

1. [1] a method for screening a compound having the activity of promoting or inhibiting the binding of the protein of the invention, a partial peptide thereof or a salt thereof to NKG2D or the activation of immunocytes (also referred to as a promoter or inhibitor in “(2) Screening for pharmaceutical candidates against diseases”), characterized in that the protein of the invention, a partial peptide thereof or a salt thereof is used (also referred to as the screening method of this invention in “(2) Screening for pharmaceutical candidates against diseases”) and [2] a kit for screening an inhibitor, characterized in the protein of the invention, a partial peptide or a salt thereof is contained (also referred to as the screening kit of the invention in “(2) Screening for pharmaceutical candidates against diseases”), and more specifically

2. [1] a method of screening an inhibitor, characterized by comparing the case (i) where a substrate is contacted with the protein of the invention, a partial peptide thereof or a salt thereof with the case (ii) where the substrate and a test compound are contacted with the protein of the invention, a partial peptide thereof or a salt thereof, and [2] a kit for screening an inhibitor, characterized in that the protein of the invention, a partial peptide thereof or a salt thereof and a substrate are contained.

Specifically, the screening method and the screening kit described above are characterized in that for example, the binding of the protein etc. of the invention to NKG2D or the activation of immunocytes is measured and compared between the cases (i) and (ii).

The binding of the protein etc. of the invention to NKG2D and the activation of immunocytes can be measured in a method known per se or its analogous method.

The test compound includes e.g. peptides, proteins, non-peptide compounds, synthetic compounds, fermentation products, cellular extracts, plant extracts, animal tissue extracts etc., and these compounds may be novel or known compounds.

The substrate may be any one which can serve as the substrate for the protein etc. of the invention.

For example, a test compound showing higher inhibition of the binding to NKG2D or the activation of immunocytes in the case (ii) by about 20% or more, preferably about 30% or more, more preferably about 50% or more than in the case (i) can be selected as a compound inhibiting the binding of the protein etc. of the invention to NKG2D or the activation of immunocytes.

Further, a test compound showing higher promotion of the binding to NKG2D or the activation of immunocytes in the case (ii) by about 20% or more, preferably about 30% or more, more preferably about 50% or more than in the case (i) can be selected as a compound promoting the binding of the protein etc. of the invention to NKG2D or the activation of immunocytes.

The screening kit of the invention comprises the protein of the invention, a partial peptide thereof or a salt thereof. For example, the screening kit of the invention is as follows:

[Screening Reagents]

1. Measurement Buffers

Phosphate buffer containing FBS (fetal bovine serum)

Tris-HCl buffer, pH 7.5 (containing MgCl₂, EDTA)

2. Protein Standard

A fusion protein between the protein of the invention, a partial peptide thereof or a salt thereof and Fc

3. KG2D Source

KG2D-expressing CHO-K1 cells

4. Labeling Antibody

Anti-human IgG (Fc)-FITC conjugate

5. Detection

Flow cytometer

[Measurement Method]

The protein standard and a test compound are added to the KG2D-expressing CHO-K₁ cells, and then labeled with the anti-human IgG (Fc)-FITC conjugate, and the binding thereof to KG2D is measured with a flow cytometer.

The compounds or salts thereof obtained by the screening method or screening kit of the invention are compounds selected from the above test compounds e.g. peptides, proteins, non-peptide compounds, synthetic compounds, fermentation products, cellular extracts, plant extracts, animal tissue extracts, plasma etc., and these are compounds having the activity of inhibiting the binding of the protein etc. of the invention to NKG2D.

As the salts of said compounds, the same salts as those of the protein of the invention described above are used.

When the compound obtained by the screening method or screening kit of the invention is used as the therapeutic or preventive agent described above, the compound can be used according to a conventional means. For example, the compound can be formed into tablets, capsules, elixirs, microcapsules, aseptic solutions and suspensions in the same manner as in production of the pharmaceutical composition containing the protein etc. of the invention described above.

The pharmaceutical composition thus obtained is safe and low toxic so that it can be administered into e.g. humans or warm-blooded animals (e.g., mouse, rat, rabbit, sheep, pig, cow, horse, bird, cat, dog, monkey etc.).

The dose of the compound or a salt thereof is varied depending on its action, the intended disease, the subject of administration, administration route etc., and when the compound having the activity of inhibiting the cell-proliferating activity of the protein etc. of the invention is orally administered for example for the purpose of treatment of autoimmune diseases, said compound is administered in a dose of usually about 0.1 to 100 mg, preferably about 1.0 to 50 mg, more preferably about 1.0 to 20 mg every day into a (60 kg) adult. In the case of parenteral administration, the dose of said compound is varied depending on the subject of administration, the intended disease etc., and when the compound having the activity of inhibiting the cell-proliferating activity of the protein etc. of the invention is administered in the form of an injection for example for the purpose of treatment of autoimmune diseases, said compound is conveniently injected intravenously in a dose of usually about 0.01 to 30 mg, preferably about 0.1 to 20 mg, more preferably about 0.1 to 10 mg every day into a (60 kg) adult. Said compound can be administered into other animals in the same dose per 60 kg as described above.

To carry out the screening method, the protein etc. of the invention is suspended in a buffer suitable for screening, whereby a preparation of the protein etc. of the invention is prepared. The buffer may be any buffers such as phosphate buffer, Tris-HCl buffer etc. at a pH value of about 4 to 10 (desirably a pH value of about 6 to 8) which do not inhibit the reaction of the protein etc. of the invention with a test compound.

(3) Quantification of the Protein or Partial Peptide of the Invention or Salts Thereof

The antibody against the protein etc. of the invention (also referred to hereinafter as the antibody of the invention) can specifically recognize the protein etc. of the invention, and thus the antibody of the invention can be used for e.g. quantification of the protein etc. of the invention in a test sample, particularly for quantification thereof by sandwich immunoassays.

That is, this invention provides:

(i) A method of quantifying the protein etc. of the invention in a test solution, which comprises allowing a test solution and the labeled protein etc. of the invention to react competitively with the antibody of the invention and determining the proportion of the labeled protein etc. of the invention bound to said antibody; and

(ii) A method of quantifying the protein etc. of the invention in a test solution, which comprises allowing a test solution to react with the antibody of the invention insolubilized on a carrier and another labeled antibody of the invention simultaneously or successively and then measuring the activity of the label on the insolubilizing carrier.

In the quantification method (ii) above, it is desired that one antibody is an antibody recognizing an N-terminal region of the protein etc. of this invention, and the other antibody is an antibody recognizing a C-terminal region of the protein etc. of the invention.

The monoclonal antibody against the protein etc. of the invention (hereinafter, also referred to as the monoclonal antibody of the invention) can be used not only for quantifying the protein etc. of the invention but also for detection thereof by tissue staining etc. For these purposes, the antibody molecule itself or an F(ab′)₂, Fab′ or Fab fraction of the antibody molecule may be used.

The method of quantifying the protein etc. of the invention by use of the antibody of the invention is not particularly limited as long as it is a method wherein the amount of the antibody, the antigen or an antigen-antibody conjugate, corresponding to the amount of the antigen (e.g., the amount of the protein) in a test solution, is detected by chemical or physical means and calculated on the basis of a standard curve prepared using standard solutions containing known amounts of the antigen. For example, nephelometry, the competitive method, the immunometric method and the sandwich method are preferably used, among which the sandwich method described later is particularly preferably used in respect of sensitivity and specificity.

As the label used in the measurement method using the labeled material, for example a radioisotope, an enzyme, a fluorescent material or a luminescent material is used. The radioisotope includes, for example, [¹²⁵I], [¹³¹I], [³H], [¹⁴C] etc. The enzyme is preferably a stable enzyme with high specific activity, which includes, for example, β-galactosidase, β-glucosidase, alkali phosphatase, peroxidase and malate dehydrogenase. The fluorescent material includes, for example, fluorescamine and fluorescein isocyanate. The luminescent material includes, for example, luminol, luminol derivatives, luciferin, and lucigenin. Further, a biotin-avidin system can also be used for binding the label to the antibody or antigen.

The antigen or antibody may be insolubilized by physical adsorption or via chemical bonding used conventionally for insolubilization or immobilization of proteins, enzymes etc. The carrier includes insoluble polysaccharides such as agarose, dextran and cellulose, synthetic resin such as polystyrene, polyacrylamide and silicon, and glass etc.

In the sandwich method, a test sample is allowed to react with the insolubilized monoclonal antibody of the invention (primary reaction), then another labeled monoclonal antibody of the invention is allowed to react therewith (secondary reaction), and the activity of the labeling agent on the insolubilizing carrier is measured, whereby the protein of the invention in the test sample can be quantitatively determined. The primary and secondary reactions may be carried out in the reverse order, simultaneously, or separately after a predetermined time. The labeling agent and the method of insolubilization may be in accordance with those described above. For the purpose of improving measurement sensitivity etc., immunoassays by the sandwich method may make use of not only one kind of antibody but also a mixture of two or more kinds of antibodies as the solid-phase antibody or labeling antibody.

In the method of measuring the protein etc. of the invention by the sandwich method according to this invention, the monoclonal antibodies of the invention used in the primary and secondary reactions are preferably those antibodies to which the protein etc. of the invention are bound at different sites. That is, when the antibody used in e.g. the secondary reaction recognizes the C-terminal region of the protein etc. of the invention, while the other antibody used in the primary reaction recognizes other regions (e.g. the N-terminal region) than the C-terminal region.

The monoclonal antibody of the invention can be used not only in the sandwich method but also in other measurement systems such as competitive method, immunometric method and nephelometry.

In the competitive method, an antigen in a test sample and its labeled antigen are allowed to react competitively with the antibody, then the unreacted labeled antigen (F) and the labeled antibody (B) bound to the antibody are separated from each other (B/F separation), and the amount of either labeled B or F is determined to quantify the amount of the antigen in the test sample. This reaction method employs either a liquid-phase method in which a soluble antibody is used as the antibody, and polyethylene glycol and a second antibody different from the above antibody are used in B/F separation, or a solid-phase method in which a solid-phase antibody is used as the primary antibody, or a soluble antibody is used as the primary antibody while a solid-phase antibody is used as the secondary antibody.

In the immunometric method, the antigen in a test solution and the solid-phase antigen are allowed to react competitively with a predetermined amount of the labeled antibody, followed by separating the solid phase from the liquid phase, or alternatively the antigen in a test solution is allowed to react with an excess of the labeled antibody, and then the solid-phase antigen is added thereto to permit the unreacted labeled antibody to be bound to the solid phase, followed by separating the solid phase from the liquid phase. Then, the amount of the label in either phase is measured to quantify the amount of the antigen in the test solution.

In nephelometry, the amount of insoluble precipitates in gel or solution, formed by antigen-antibody reaction, is measured. Laser nephelometry using laser scattering can be applied preferably to the case where precipitates are obtained in a small amount because of a very small amount of the antigen in a test solution.

For application of these immunoassays to the quantification method of the invention, it is not necessary to establish special conditions, procedures etc. A measurement system for the protein etc. of the invention can be established in an ordinary manner by those skilled in the art by modifying the conventional conditions and procedures. The details of these general technical means can be referred to in general remarks, books etc.

For example, reference can be made to “Radioimmunoassays” edited by Hirhoshi Irie (published by Kodansha in 1974), “Radioimmunoassays” (2nd edition) edited by Hiroshi Irie (published by Kodansha in 1979), “Enzyme Immunoassays” edited by Eiji Ishikawa (published by Igakushoin in 1978), “Enzyme Immunoassays” (2nd edition) edited by Eiji Ishikawa (published by Igakushoin in 1982), “Enzyme Immunoassays” (3rd edition) edited by Eiji Ishikawa (published by Igakushoin in 1987), Methods in Enzymology, Vol. 70 (Immunochemical Techniques (Part A), Methods in Enzymology, Vol. 73 (Immunochemical Techniques (Part B)), Methods in Enzymology, Vol. 74 (Immunochemical Techniques (Part C)), Methods in Enzymology, Vol. 84 (Immunochemical Techniques (Part D: Selected Immunoassays)), Methods in Enzymology, Vol. 92 (Immunochemical Techniques (Part E: Monoclonal Antibodies and General Immunoassay Methods)), and Methods in Enzymology, Vol. 121 (Immunochemical Techniques (Part I: Hybridoma Technology and Monoclonal Antibodies)) (which are published by Academic Press Ltd.).

By using the antibody of the invention in the manner as described above, the protein etc. of the invention can be quantified with good sensitivity.

When a reduction in the protein etc. of the invention is detected by quantifying the concentration of the protein etc. of the invention by using the antibody of the invention, patients examined can be diagnosed as having various cancers (for example, uterine cancer, endometrium tumor, breast cancer, colon cancer, prostate cancer, lung cancer, kidney cancer, neuroblastoma, bladder cancer, melanoma etc.), or it is predicted that they will probably have such diseases.

Further, the antibody of the invention can be used to detect the protein etc. of the invention present in test samples such as body fluids and tissues. The antibody of the invention can also be used for preparation of an antibody column used in purification of the protein etc. of the invention, for detection of the protein etc. of the invention in each fraction during purification, and for analysis of the behavior of the protein etc. of the invention in cells examined.

The antibody of the invention having the action of neutralizing the activity of the protein etc. of the invention (neutralizing antibody) can be used for example as an immunosupressant or an antiinflammatory agent not only against various diseases such as autoimmune diseases and infectious hyper-immunoreactions after transplantation of organs and tissues.

Hereinafter, the neutralizing antibody of the invention in “(4) Pharmaceutical composition containing the antibody of the invention” is also referred to as the antibody of the invention.

The therapeutic or preventive agent containing the antibody of the invention against the diseases described above can be administered orally or parenterally as a liquid agent directly or as a pharmaceutical composition in a suitable form into humans or mammals (e.g., rat, rabbit, sheep, pig, cow, cat, dog, monkey etc.). The dose is varied depending on the subject of administration, intended disease, conditions, administration route etc., but for example, the antibody of the invention is conveniently injected intravenously once to about 5 times preferably once to about 3 times every day, in a single dose of usually about 0.01 to 20 mg/kg, preferably about 0.1 to 10 mg/kg, more preferably 0.1 to 5 mg/kg, to treat and prevent autoimmune diseases for adults. A similar dose can be administered in other parenteral administration or oral administration. In the case of particularly severe conditions, the dose may be increased depending on the conditions.

The antibody of the invention can be administered as such or as a suitable pharmaceutical composition. The pharmaceutical composition used in the administration described above contains the above antibody or salts thereof, pharmaceutically acceptable carriers, diluents or excipients. The composition is provided in a pharmaceutical form suitable for oral or parenteral administration.

That is, the composition for e.g. oral administration is in a solid or liquid form, specifically in the form of tablets (including sugar-coated tablets and film-coated tablets), pills, granules, powders, capsules (including soft capsules), syrups, emulsions, suspensions etc. The composition is produced in a method known per se and contains carriers, diluents or excipients generally used in the field of pharmaceutical manufacturing. For example, the carriers and excipients used in tablets include lactose, starch, sucrose, magnesium stearate etc.

The composition used for parenteral administration includes e.g. injections, suppositories etc., and the injections are in pharmaceutical forms such as intravenous injection, subcutaneous injection, intracutaneous injection, intramuscular injection and intravenous infusion. These injections are prepared in a usual manner by dissolving, suspending or emulsifying the antibody or its salt in a germ-free, aqueous or oily solution used conventionally in injections. The aqueous solution for injection includes e.g. physiological saline or an isotonic solution containing glucose and other auxiliary chemicals, and may be used in combination with suitable solubilizers such as alcohols (e.g., ethanol etc.), polyalcohols (e.g., propylene glycol, polyethylene glycol) and non-ionic surfactants (e.g., Polysorbate 80™ and HCO-50 (i.e. polyoxyethylene (50 mol) adduct of hydrogenated castor oil). The oily solution includes e.g. sesame oil, soybean oil etc., and may be used in combination with solubilizers such as benzyl benzoate, benzyl alcohol etc. The prepared injection is introduced usually into suitable ampoules. The suppositories used in administration into rectum are prepared by mixing the antibody or its salt with a conventional base for suppositories.

The oral or parenteral pharmaceutical composition described above is produced conveniently in a unit form adapted to the prescribed dose of the active ingredient. The pharmaceutical composition in such a unit form includes e.g. tablets, pills, capsules, injections (ampoules) and suppositories, where the antibody is contained in an amount of usually 5 to 500 mg/dose in these pharmaceutical forms, preferably 5 to 100 mg/dose in the injections or 10 to 250 mg/dose in other pharmaceutical forms.

The respective compositions described above may contain any other active ingredients that do not generate undesired interaction upon incorporation into the antibody.

When bases or amino acids are expressed in abbreviations in this specification and drawings, the following abbreviations are used in accordance with Commission on Biochemical Nomenclature IUPAC-IUB. If amino acids can occur as optical isomers, L-isomers are referred to unless otherwise specified.
DNA: deoxyribonucleic acid
cDNA: complementary deoxyribonucleic acid
A: adenine
T: thymine
G: guanine
C: cytosine
I: inosine
R: adenine (A) or guanine (G)
Y: thymine (T) or cytosine (C)
M: adenine (A) or cytosine (C)
K: guanine (G) or thymine (T)
S: guanine (G) or cytosine (C)
W: adenine (A) or thymine (T)
B: guanine (G), guanine (G) or thymine (T)
D: adenine (A), guanine (G) or thymine (T)
V: adenine (A), guanine (G) or cytosine (C)
RNA: ribonucleic acid
mRNA: messenger ribonucleic acid
dATP: deoxyadenosine triphosphate
dTTP: deoxythymidine triphosphate
dGTP: deoxyguanosine triphosphate
dCTP: deoxycytidine triphosphate
ATP: adenosine triphosphate
Gly: glycine
Ala: alanine
Val: valine
Leu: leucine
Ile: isoleucine
Ser: serine
Thr: threonine
Cys: cysteine
Met: methionine
Glu: glutamic acid
Asp: aspartic acid
Lys: lysine
Arg: arginine
His: histidine
Phe: phenyl alanine
Tyr: tyrosine
Trp: tryptophan
Pro: proline
Asn: asparagine
Gln: glutamine
pGlu: pyroglutamic acid

The substituents, protective groups and reagents appearing frequently in this specification are expressed in the following symbols.
Me: methyl group
Et: ethyl group
Bu: butyl group
Ph: phenyl group
TC: thiazolidine-4 (R)-carboxamide group
Tos: p-toluene sulfonyl
CHO: formyl
Bzl: benzyl
Cl₂-Bzl: 2,6-dichlorobenzyl
MBzl: methoxybenzyl
MeBzl: 4-methylbenzyl
OcHex: cyclohexyl ester
OBzl: benzyl ester
Bom: benzyloxymethyl
Z: benzyloxycarbonyl
Cl—Z: 2-chlorobenzyloxycarbonyl
Br—Z: 2-bromobenzyloxycarbonyl
Boc: t-butoxycarbonyl
DNP: dinitrophenyl
Trt: trityl
Bum: t-butoxymethyl
Fmoc: N-9-fluorenyl methoxycarbonyl
HOBt: 1-hydroxybenztriazole
HOOBt: 3,4-dihydro-3-hydroxy-4-oxo-1,2,3-benzotriazine
HONB: 1-hydroxy-5-norbornene-2,3-dicarboximide
DCC: N,N′-dicyclohexyl carbodiimide
DMF: N,N-dimethylformamide
TEA: triethylamine
WSCD: 1-ethyl-3-(3-dimethylaminopropyl)-carbodiimide
EDTA: ethylene diamine tetraacetate
SDS: sodium dodecyl sulfate

The sequence numbers in the Sequence Listing in this specification show the following sequences:

SEQ ID NO:1 shows the nucleotide sequence of the primer (synthetic) DNA used in Example 1.

SEQ ID NO:2 shows the nucleotide sequence of the primer (synthetic) DNA used in Example 1.

SEQ ID NO:3 shows the nucleotide sequence of DNA coding for the human-derived protein of the invention having the amino acid sequence set forth in SEQ ID NO:4.

SEQ ID NO: 4 shows the amino acid sequence of the human-derived protein (CSP2 protein) of the invention.

SEQ ID NO:5 shows the nucleotide sequence of the primer (synthetic) DNA used in Example 2.

SEQ ID NO:6 shows the nucleotide sequence of the primer (synthetic) DNA used in Example 2.

SEQ ID NO:7 shows the FLAG sequence.

SEQ ID NO:8 shows the nucleotide sequence of the primer (synthetic) DNA used in Example 6.

SEQ ID NO:9 shows the nucleotide sequence of the primer (synthetic) DNA used in Example 6.

SEQ ID NO:10 shows the nucleotide sequence of the primer (synthetic) DNA used in Example 6.

SEQ ID NO:11 shows the nucleotide sequence of the primer (synthetic) DNA used in Example 7.

SEQ ID NO:12 shows the nucleotide sequence of the primer (synthetic) DNA used in Example 7.

The transformant Escherichia coli JM109/pCR2.1-CSP2 obtained in Example 1 below has been deposited under FERM BP-7091 from Mar. 16, 2000 with International Patent Organism Depositary (IPOD), National Institute of Advanced Industrial Science and Technology (AIST), Chuo No. 6, Higashi 1-1-1, Tsukuba City, Ibaraki Pref., JP, and under IFO 16363 from Feb. 16, 2000 with Institute for Fermentation, Osaka (IFO), Juso-honmachi 2-17-85, Yodogawa-ku, Osaka City, Osaka Pref., JP.

Hereinafter, this invention is described in more detail by reference to the Examples, which are not intended to limit this invention. The genetic manipulation using E. coli was conducted in accordance with methods described in Molecular Cloning.

### EXAMPLE 1

### Cloning of CSP2 cDNA

PCR was conducted using human kidney cDNA (Marathon-Ready™ cDNA; Clontech) as the template and two primers i.e. primer 1 (SEQ ID NO:1) and primer 2 (SEQ ID NO:2). Using Advantage 2 Polymerase Mixture (Clontech), the PCR reaction was carried out (1) at 95° C. for 1 minute and then (2) 30 times at 95° C. for 30 seconds, 62° C. for 30 seconds, and 68° C. for 2 minutes, followed by (3) extension reaction at 68° C. for 5 minutes. After the reaction, the reaction product was cloned into plasmid vector pCR2.1 (Invitrogen) according to a protocol of TA Cloning Kit (Invitrogen). As a result of analysis of each clone sequence, a cDNA sequence (SEQ ID NO:3) coding for a novel secretory protein was obtained. An amino acid sequence (SEQ ID NO:4) deduced from this cDNA was designated CSP2. A transformant harboring the cDNA shown in SEQ ID NO:3 was designated Escherichia coli JM109/pCR2.1-CSP2.

### EXAMPLE 2

### Construction of an Expression Vector for Expressing CSP2 in Animal Cells

An expression vector for expressing CSP2 in animal cells was obtained by inserting a DNA fragment containing an open reading frame (ORF) coding for CSP2 into an animal cell expression vector pCAN618FLAG. pCAN618FLAG is derived from plasmid vector pCAN618 (International Application; PCT JP00/05685), in which the desired protein can be expressed as a FLAG fusion protein by ligating the ORF to a nucleotide sequence coding for 8-amino-acid FLAG sequence (SEQ ID NO:7; Asp-Tyr-Lys-Asp-Asp-Asp-Asp-Lys) present just after Sal I site and a termination codon. First, PCR was conducted using the CSP2-coding cDNA as the template and a synthetic DNA (SEQ ID NO:5) designed to have a restriction enzyme Mfe I recognition site just before a translation initiation codon and a synthetic DNA (SEQ ID NO:6) designed to have a restriction enzyme Sal I recognition site after amino acid 222 in CSP2 protein. Using Advantage 2 Polymerase Mixture (Clontech), the PCR reaction was carried out (1) at 94° C. for 1 minute and then (2) 30 times at 98° C. for 10 seconds, 60° C. for 30 seconds, and 72° C. for 1 minute, followed by (3) extension reaction at 72° C. for 10 minutes, whereby a DNA fragment containing the ORF for CSP2 was obtained. This DNA fragment was cloned into plasmid vector pVR2.1-TOPO (Invitrogen) according to a protocol of TA Cloning Kit (Invitrogen). The resultant plasmid was cleaved with restriction enzymes Mfe I and Sal I, and the inserted fragment was recovered and inserted into between Eco RI/Sal I sites in pCAN618FLAG, to give an expression vector pCAN618/CSP2-FLAG for expressing human CSP2 protein in animal cells.

### EXAMPLE 3

### Expression of CSP2 in COS-7 Cells

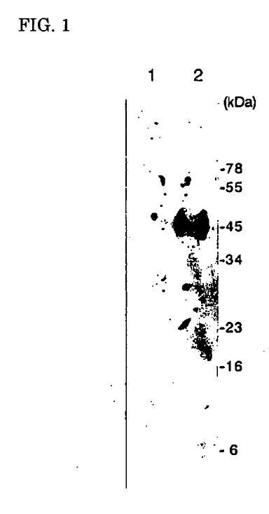

2×10⁶ COS-7 cells were cultured for 24 hours in DMEM (medium; Gibco BRL) containing 10% FBS (fetal bovine serum) in a 10-cm Petri dish. The expression vector pCAN618/CSP2-FLAG obtained in Example 2 or the control vector pCAN618 was introduced by Lipofect AMINE (Gibco BRL) into the cells, and the cells were further cultured for 18 hours. Then, the medium was exchanged with Opti-MEM (medium; Gibco BRL) containing 0.05% CHAPS, and the cells were further cultured for 24 hours, and the culture supernatant was recovered. After the suspended cells were removed by centrifugation, 1 μl SDS-sample buffer containing 2-mercaptoethanol was added to 1 μl of the supernatant which was then electrophoresed on 16% Peptide-PAGE (TEFCO) and transferred electrically onto a PVDF membrane (Amersham). As the primary antibody, an anti-FLAG antibody (mouse IgG; Sigma) was used, and as the secondary antibody, HRP (horseradish peroxidase)-labeled anti-mouse IgG antibody (Amersham) was used. Coloration was performed under light exposure for 5 minutes by using an ECL plus Western Blot Detection System (Amersham). The results are shown in FIG. 1. In FIG. 1, lane 2 is the expression vector (pCAN618/CSP2-FLAG), and lane 1 is the control plasmid (pCAN618). As is evident from FIG. 1, the product recognized by anti-FLAG antibody was confirmed in the culture supernatant.

### EXAMPLE 4

### Purification of CSP2 Protein from a Culture Supernatant of COS7 Cells

Because it was confirmed in Example 3 that CSP2 protein is secreted into the culture supernatant of COS7 cells, CSP2-FLAG protein was purified from the culture supernatant of COS7 cells. According to the method in Example 3, the expression vector obtained in Example 2 was introduced into COS7 cells in thirty 10-cm Petri dishes, and the culture supernatant was recovered. The recovered culture supernatant was centrifuged to remove the cells etc., and then adsorbed onto anti-FLAG M2-agarose affinity gel (SIGMA), and the objective protein was eluted with FLAG peptide (SEQ ID NO:7; Asp-Tyr-Lys-Asp-Asp-Asp-Asp-Lys). This eluate was dialyzed against TBS buffer (pH 7.2), concentrated to a volume of 500 μl by Centricon-10 (Amicon), to give a purified preparation. This sample was subjected to SDS-PAGE and subsequent silver staining, whereby a broad band was detected at a position of about 40 kDa. Further, this band was quantified for its protein by Micro BCA Protein Assay Kit (Piercr), to give 15 μl protein.

### EXAMPLE 5

### Determination of an N-terminal Amino Acid Sequence of the Purified CSP2-FLAG Protein

20 μl of the purified preparation obtained in Example 4 was diluted with 0.1% TFA and adsorbed onto a PVDF membrane to remove low-molecular contaminants. The purified preparation was analyzed in PL-Prosorb cycle by a protein sequencer Procice 491 cLC (Applied Biosystems). As a result, the amino acid residues 1. histidine (0.92 pmol), 2. serine (1.00 pmol) and 3. leucine (1.87 pmol) were obtained as major products in this order from the N-terminal, and the amino acid residues 1. glycine (0.70 pmol), 2. histidine (0.43 pmol), 3 serine (0.64 pmol) and 4. leucine (1.11 pmol) were obtained as second components in this order from the N-terminal. From this result, it was found that a N-terminal signal sequence of 30 or 29 amino acid residues is cleaved off from a precursor protein of CSP2 protein, and the resulting protein beginning at the histidine residue 31 or the glycine residue 30 is secreted as mature CSP2 protein into the medium.

### EXAMPLE 6

### Expression of a Fusion Protein of CSP2 Protein with Human IgG Fc Region

An expression vector for expressing CSP2 as a fusion protein with human IgG Fc region was constructed in the following procedure. First, PCR was carried out using human spleen IgG Fc cDNA (Clontech) as the template and synthetic DNAs (SEQ ID NOS:8 and 9) having a restriction enzyme Xho I or Not I site as an anchor. Using Advantage 2 Polymerase Mixture (Clontech), the PCR reaction was carried out (1) at 94° C. for 1 minute and then (2) 35 times at 96° C. for 10 seconds, 60° C. for 30 seconds and 72° C. for 1 minute, followed by (3) extension reaction at 72° C. for 10 minutes, whereby a DNA fragment coding for human IgG Fc region was obtained. This DNA fragment was cloned into plasmid vector pCR2.1-TOPO (Invitrogen) according to a protocol of TA Cloning Kit (Invitrogen). The resultant plasmid was cleaved with restriction enzymes Xho I and Not I, and the inserted fragment was recovered and inserted into between Xho I/Not I sites in pCAN618, to give pCAN618Fc. Then, PCR was carried out using the cDNA coding for CSP2 protein as the template and the synthetic DNA (SEQ ID NO:5) and a synthetic DNA (SEQ ID NO:10) designed to have a restriction enzyme Xho I recognition site after amino acid 221 in CSP2 protein. Using Advantage 2 Polymerase Mixture (Clontech), the PCR reaction was carried out (1) at 94° C. for 1 minute and then (2) 25 times at 98° C. for 10 seconds, 60° C. for 30 seconds and 72° C. for 1 minute, followed by (3) extension reaction at 72° C. for 10 minutes, whereby a DNA fragment containing the ORF for CSP2 was obtained. This DNA fragment was cleaved with restriction enzymes Mfe I and Xho I, recovered and inserted into between Eco RI/Xho I sites in pCAN618Fc, to give the expression vector pCAN618/CSP2-Fc for expressing human CSP2 protein in animal cells.

The resultant expression vector pCAN618/CSP2-Fc was introduced into COS7 cells in two 10-cm Petri dishes in the same manner as in Example 4, and the culture supernatant was recovered. The recovered culture supernatant was centrifuged to remove cells etc. and concentrated 100-fold by Centricon-10 (Amicon). Expression of the protein was confirmed by electrophoresis, transfer onto a PVDF membrane and coloration in the same manner as in Example 3 (the result is not shown).

### EXAMPLE 7

### Establishment of Human NKG2D-Expressing CHO-K1 Cell Strain

An expression vector for expressing human NKG2D was constructed in the following manner. First, PCR was carried out using human spleen NKG2D cDNA (Clontech) as the template and synthetic DNAs (SEQ ID NOS: 11 and 12) having a restriction enzyme Eco RI or Not I site as an anchor. Using Advantage 2 Polymerase Mixture (Clontech), the PCR reaction was carried out (1) at 95° C. for 1 minute, then (2) 5 times at 95° C. for 20 seconds and 72° C. for 4 minutes, (3) 5 times at 95° C. for 20 seconds and 68° C. for 4 minutes, (4) 30 times at 95° C. for 20 seconds, 64° C. for 20 seconds and 68° C. for 4 minutes, followed by (5) extension reaction at 68° C. for 3 minutes, whereby a DNA fragment coding for human NKG2D was obtained. This DNA fragment was cloned into plasmid vector pCR2.1-TOPO (Invitrogen) according to a protocol of TA Cloning Kit (Invitrogen). The resultant plasmid was cleaved with restriction enzymes Eco RI and Not I, and the inserted fragment was recovered and inserted into between Eco RI/Not I sites in pCAN618, to give pCAN618/hNKG2D.

The resultant expression vector pCAN618/hNKG2D was introduced into CHO-K1 cells by using Lipofect AMINE (Gibco BRL). After the introduction, the cells into which the expression vector had been introduced were selected in the presence of 0.5 mg/ml Geneticin (Wako Pure Chemical Industries, Ltd.), to give CHO-K1 cell strain CHO-K1/hNKG2D-11 expressing human NKG2D.

### EXAMPLE 8

### Binding of CSP2-Fc Protein to the Human NKG2D-Expressing CHO-K1 Cells

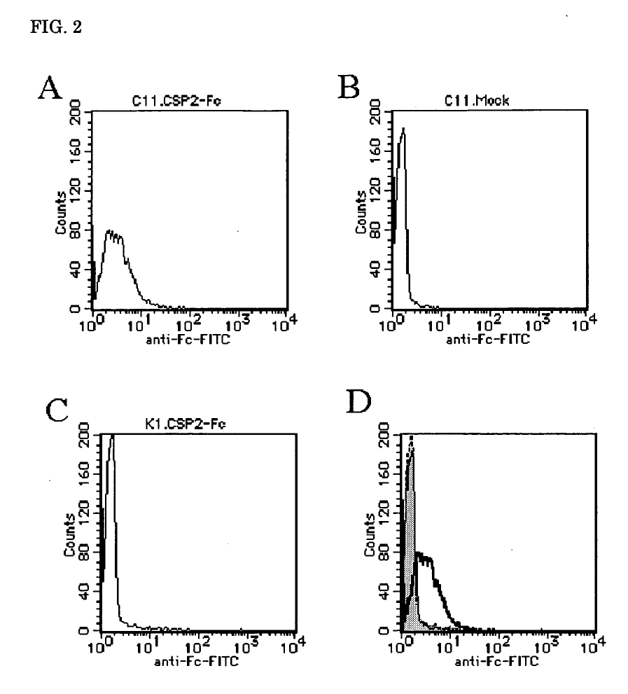

The human NKG2D-expressing CHO-K1 cell strain CHO-K1/hNKG2D-11 obtained in Example 7 was washed twice with PBS/1% FBS and suspended in 50 μl PBS/1% FBS containing 10 μl concentrate of the culture supernatant of the CSP2-Fc-expressing COS7 cells obtained in Example 6. The suspension was reacted at 0° C. for 60 minutes for binding them, then washed twice with 200 μl PBS/1% FBS, and suspended in 50 μl PBS/1% FBS containing 1 μl anti-human IgG (Fc)-FITC conjugate (Caltag). The suspension was reacted at 0° C. for 60 minutes for labeling, then washed twice with 200 μl PBS/1% FBS, and suspended again in 600 μl PBS/1% FBS. When the suspension was analyzed with a flow cytometer FACS Vantage (Becton Dickinson), cells with high intensity of FITC fluorescence were recognized, and the evident binding of CSP2-Fc protein to human NKG2D expressed on the surface of CHO-K1 cells was observed (FIG. 2A).

The binding (FIG. 2B), to CHO-K1 cells (control), of the concentrate of the culture supernatant of the CSP2-Fc-expressing COS7 cells obtained in Example 6, and the binding (FIG. 2C), to the above CHO-K1/hNKG2D-11, of a concentrate of a culture supernatant of a Mock plasmid-containing COS7 cells obtained by introducing a Mock plasmid into the cells in the same manner as in Example 6 was examined in the same manner.

The CSP2-Fc bound to the cells was detected by staining with FITC-labeled anti-human IgG (Fc) antiserum and subsequent FACS.

Evident enhancement of fluorescence intensity was observed upon addition, to the CHO-K1 cells expressing human NKG2D, of the culture supernatant concentrate of the CSP2-Fc-expressing COS7 cells (FIG. 2A) as compared with the culture supernatant concentrate of the Mock plasmid-containing COS7 cells (FIG. 2B). When the control CHO-K1 cells not expressing NKG2D was used, there was no enhancement of fluorescence intensity by CSP2-Fc (FIG. 2C). The graphs A to C are collectively shown in FIG. 2D. From these results, CSP2-Fc was revealed to bind to NKG2D expressed on the surface of the CHO-K1 cells, and NKG2D was found to be a receptor specific to CSP2 protein.

Industrial Applicability

The protein etc. of the invention have e.g. the activity of biding to NKG2D and the action of activating immunocytes, and are thus useful as an agent for treating and preventing various cancers (for example, uterine cancer, endometrium tumor, breast cancer, colon cancer, prostate cancer, lung cancer, kidney cancer, neuroblastoma, bladder cancer, melanoma etc.). Further, the protein of the invention is useful as a reagent for screening a compound or a salt thereof promoting or inhibiting the activity of the protein of the invention, and an inhibitor obtained by screening is expected to act as an immunosupressant and an antiinflammatory agent.

Further, the antibody against the protein of the invention can specifically recognize the protein of the invention and is thus usable to quantify the protein of the invention in a sample and usable as a diagnostic agent for the various cancers described above. Further, the humanized antibody of the invention can be used as an immunosupressant or an antiinflammatory agent.

Sequence Listing Free Text

SEQ ID NO: 1

Designed oligonucleotide primer to amplify DNA encoding CSP2

SEQ ID NO:2

Designed oligonucleotide primer to amplify DNA encoding CSP2

SEQ ID NO:5

Designed oligonucleotide primer to amplify DNA encoding CSP2

SEQ ID NO:6

Designed oligonucleotide primer to amplify DNA encoding CSP2

SEQ ID NO:7

FLAG sequence of pCAN618FLAG

SEQ ID NO:8

Designed oligonucleotide primer to amplify DNA encoding IgG Fc

SEQ ID NO:9

Designed oligonucleotide primer to amplify DNA encoding IgG Fc

SEQ ID NO: 10

Designed oligonucleotide primer to amplify DNA encoding CSP2

SEQ ID NO:11

Designed oligonucleotide primer to amplify DNA encoding NKG2D

SEQ ID NO:12

Designed oligonucleotide primer to amplify DNA encoding NKG2d

1. An isolated protein comprising an amino acid sequence identical or substantially identical with the amino acid sequence set forth in SEQ ID NO:4 or a salt thereof:

2. A partial peptide of the protein according to claim 1, or an amide, an ester or a salt thereof.

3. An isolated DNA comprising a DNA coding for the protein according to claim 1.

4. The DNA according to claim 3, which comprises the nucleotide sequence set forth in SEQ ID NO:3.

5. An isolated DNA comprising a DNA coding for the partial peptide according to claim 2.

6. A recombinant vector comprising a DNA encoding for the protein of claim 1 or a partial peptide thereof.

7. A transformant transformed with the recombinant vector according to claim 6.

8. A process for producing a protein according to claim 1, the partial peptide or an amide, an ester or a salt thereof, which comprises transforming a host cell with an expressible recombinant vector comprising a nucleic acid which encodes for said protein, culturing the transformant under conditions suitable for expression of said protein, and obtaining the protein of claim 1, or the partial peptide thereof.

9. An antibody against the protein according to claim 1 or a salt thereof, or the partial peptide thereof or an amide, an ester or a salt thereof.

10. A diagnostic composition comprising an DNA which encodes for the protein of claim 1, or a partial peptide thereof, or an antibody which binds specifically to a protein of claim 1 or a partial peptide thereof.

11. A pharmaceutical composition comprising the protein according to claim 1 or a salt thereof, or the partial peptide thereof or an amide, an ester or a salt thereof.

12. A pharmaceutical composition according to claim 11, for preventing and treating cancers.

13. A method of screening a compound or a salt thereof for promoting or inhibiting an activity of the protein according to claim 1 or a salt thereof, or the partial peptide thereof or an amide, an ester or a salt thereof, which comprises contacting a test compound with the protein according to claim 1 or a salt thereof, or the partial peptide thereof or an amide thereof, an ester thereof or a salt thereof in a screening assay and detecting change in activity of said protein as compared with a baseline control.

14. A kit for screening a compound or a salt thereof promoting or inhibiting an activity of the protein according to claim 1 or a salt thereof, or the partial peptide thereof or an amide, an ester or a salt thereof, which comprises the protein according to claim 1 or a salt thereof, or the partial peptide thereof or an amide thereof, an ester thereof or a salt thereof and instructions for screening.

15. A compound or a salt thereof promoting an activity of the protein comprising an amino acid sequence identical or substantially identical with the amino acid sequence set forth in SEQ ID NO:4 or a salt thereof, or the partial peptide thereof or an amide, an ester or a salt thereof, which is obtainable by the screening method according to claim 13.

16. A pharmaceutical composition comprising the compound according to claim 15 or a salt thereof and a pharmaceutically acceptable carrier, diluent or excipient.

17. A method for preventing or treating cancer in a mammal in need thereof comprising administering an effective amount of a pharmaceutical composition according to claim 16, to said mammal.

18. A method for making a pharmaceutical composition having an anticancer action comprising combining (i) an isolated protein comprising an amino acid sequence identical or substantially identical with the amino acid sequence set forth in SEQ ID NO:4 or a salt thereof, or the partial peptide thereof or an amide, an ester or a salt thereof, or (ii) a compound or a salt thereof promoting an activity of said isolated protein comprising an amino acid sequence identical or substantially identical with the amino acid sequence set forth in SEQ ID NO:4 or a salt thereof, or the partial peptide thereof or an amide, an ester or a salt thereof, which is obtained by the screening method according to claim 13, with a pharmaceutically acceptable carrier, diluent or excipient.

19. A method of preventing and treating cancer, which comprises administering to mammals (i) the protein comprising an amino acid sequence identical or substantially identical with the amino acid sequence set forth in SEQ ID NO:4 or a salt thereof, or the partial peptide thereof or an amide, an ester or a salt thereof, or (ii) the compound or a salt thereof promoting an activity of the protein comprising an amino acid sequence identical or substantially identical with the amino acid sequence set forth in SEQ ID NO:4 or a salt thereof, or the partial peptide thereof or an amide, an ester or a salt thereof, which is obtained by the screening method according to claim 13.

20. A compound or a salt thereof inhibiting an activity of the protein comprising an amino acid sequence identical or substantially identical with the amino acid sequence set forth in SEQ ID NO:4 or a salt thereof, or the partial peptide thereof or an amide, an ester or a salt thereof, which is obtained by the screening method according to claim 13.

21. A pharmaceutical composition comprising the compound according to claim 20 or a salt thereof and a pharmaceutically acceptable carrier, diluent or excipient.

22. The pharmaceutical composition according to claim 21, which is an immunosupressant or an antiinflammatory composition.

23. A method for making a pharmaceutical composition having an immunosuppressive or anti-inflammatory action comprising combining (i) a compound or a salt thereof which inhibits the activity of the a protein comprising an amino acid sequence identical or substantially identical with the amino acid sequence set forth in SEQ ID NO:4 or a salt thereof, or the partial thereof or an amide, an ester or a salt thereof, which is obtained by the screening method according to claim 13, or (ii) an antibody which binds specifically thereto with a pharmaceutically acceptable carrier, diluent or excipient.

24. A method of immunorepression or a method of treating inflammations, which comprises administering to mammals the compound or a salt thereof inhibiting the activity of the protein comprising an amino acid sequence identical or substantially identical with the amino acid sequence set forth in SEQ ID NO:4 or a salt thereof, or the partial peptide thereof or an amide thereof, an ester thereof or a salt thereof, which is obtained by the screening method according to claim 13, or an antibody which binds specifically thereto.


SEQUENCE LISTING (12 sequences)

SEQ ID NO: 1 - DNA, 30 bp, Artificial Sequence
  Designed oligonucleotide primer to amplify DNA encoding CSP2
ctcgagacgcccagcttcctgcctgttact

SEQ ID NO: 2 - DNA, 30 bp, Artificial Sequence
  Designed oligonucleotide primer to amplify DNA encoding CSP2
ctcgaggtgggagccaaggctgtcagcgat

SEQ ID NO: 3 - DNA, 765 bp, Human
atgcgaagaatatccctgacttctagccctgtgcgccttcttttgtttctgctgttgctactaatagccttggagatcatggttggtggtcactctctttgcttcaacttcactataaaatcattgtccagacctggacagccctggtgtgaagcgcaggtcttcttgaataaaaatcttttccttcagtacaacagtgacaacaacatggtcaaacctctgggcctcctggggaagaaggtaaatgccaccagcacttggggagaattgacccaaacgctgggagaagtggggcgagacctcaggatgctcctttgtgacatcaaaccccagataaagaccagtgatccttccactctgcaagtcgagatgttttgtcaacgtgaagcagaacggtgcactggtgcatcctggcagttcgccatcaatggagagaaatccctcctctttgacgcaatgaacatgacctggacagtaattaatcatgaagccagtaagatcaaggagacatggaagaaagacagagggctggaaaagtatttcaggaagctctcaaagggagactgcgatcactggctcagggaattcttagggcactgggaggcaatgccagaaccgacagtgtcaccagtaaatgcttcagatatccactggtcttcttctagtctaccagatagatggatcatcctgggggcattcatcctgttacttttaatgggaattgttctcatctgtgtctggtggcaaaatggcagaagatccacc

SEQ ID NO: 4 - protein, 255 aa, Human
MRRISLTSSPVRLLLFLLLLLIALEIMVGGHSLCFNFTIKSLSRPGQPWCEAQVFLNKNLFLQYNSDNNMVKPLGLLGKKVNATSTWGELTQTLGEVGRDLRMLLCDIKPQIKTSDPSTLQVEMFCQREAERCTGASWQFAINGEKSLLFDAMNMTWTVINHEASKIKETWKKDRGLEKYFRKLSKGDCDHWLREFLGHWEAMPEPTVSPVNASDIHWSSSSLPDRWIILGAFILLLLMGIVLICVWWQNGRRST

SEQ ID NO: 5 - DNA, 35 bp, Artificial Sequence
  Designed oligonucleotide primer to amplify DNA encoding CSP2
caattgccaccatgcgaagaatatccctgacttct

SEQ ID NO: 6 - DNA, 31 bp, Artificial Sequence
  Designed oligonucleotide primer to amplify DNA encoding CSP2
gtcgacactagaagaagaccagtggatatct

SEQ ID NO: 7 - protein, 8 aa, Artificial Sequence
  FLAG sequence of pCAN618FLAG
DYKDDDDK

SEQ ID NO: 8 - DNA, 30 bp, Artificial Sequence
  Designed oligonucleotide primer to amplify DNA encoding IgG Fc
ctcgagatcttgtgacaaaactcacacatg

SEQ ID NO: 9 - DNA, 30 bp, Artificial Sequence
  Designed oligonucleotide primer to amplify DNA encoding IgG Fc
gcggccgctcatttacccggagacagggag

SEQ ID NO: 10 - DNA, 35 bp, Artificial Sequence
  Designed oligonucleotide primer to amplify DNA encoding CSP2
gtagactcgaggaagaccagtggatatctgaagca

SEQ ID NO: 11 - DNA, 42 bp, Artificial Sequence
  Designed oligonucleotide primer to amplify DNA encoding NKG2D
atgaattccaccatggggtggattcgtggtcggaggtctcga

SEQ ID NO: 12 - DNA, 38 bp, Artificial Sequence
  Designed oligonucleotide primer to amplify DNA encoding NKG2D
aagcggccgcttacacagtcctttgcatgcagatgtag